(19) United States
(12) Patent Application Publication
(10) Pub. No.: US 2002/0160988 A1
(43) Pub. Date: Oct. 31, 2002

(54) Compounds co-inducing cholinergic up-regulation and inflammation down-regulation and uses thereof

(75) Inventors: Gabriel Amitai, Rehovot (IL); Rachel Adani, Moshav Gealia (IL); Ishai Rabinovitz, Nes Ziona (IL); Gali Sod-Moriah, Rehovot (IL); Haim Meshulam, Bat Yam (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Israel Institute for Biological Research

(21) Appl. No.: 09/906,952

(22) Filed: Jul. 16, 2001

Related U.S. Application Data
(60) Provisional application No. 60/269,343, filed on Feb. 20, 2001.

(51) Int. Cl. A61K 31/695; A61K 31/675; A61K 31/415

(52) U.S. Cl. 514/159; 514/94; 514/63; 514/406

(57) ABSTRACT

Chimeric compounds are disclosed which are covalent conjugates of reversible or irreversible cholinergic up-regulators and non-steroidal anti-inflammatory drugs (NSAIDs), methods for their synthesis and use thereof for treatment and/or prevention of central nervous system (CNS) disorders and diseases.

This application claims the benefit of priority from U.S. Provisional Patent Application No. 60/269,343, filed Feb. 20, 2001.

### FIELD AND BACKGROUND OF THE INVENTION

The present invention relates to preparation and use of novel bifunctional chimeric compounds for the treatment of central nervous system (CNS) disorders and diseases. More particularly, the present invention relates to novel chimeric compounds which are conjugates of reversible or irreversible cholinergic up-regulators and non-steroidal anti-inflammatory drugs (NSAIDs) and their use in the treatment of various CNS disorders and diseases such as Alzheimer's disease (AD), cerebral ischemia or stroke and closed head injury.

The development of new drugs for treatment of Alzheimer's disease (AD) is presently known to take three possible directions: (i) the development of cholinergic up-regulators, which includes compounds such as cholinesterase inhibitors (ChEI), cholinergic M1 agonists, M2 antagonists and nicotinic agonists; (ii) the development of compounds which decrease the level of beta amyloid peptide ((β-β4) such as amyloid precursor protein (APP) releasers, β-A4 processors and anti-aggregation agents for the reduction of deposited β-A4 level in the brain; and (iii) the use of anti-inflammatory drugs, free radical scavengers and antioxidants for the correction of the inflammatory response and free radical formation which occur during the progress of the disease (1).

Nevertheless, the only clinically used drugs, which presently demonstrate efficacy in AD treatment and the only AD drugs approved by the FDA are ChEIs (e.g., ARICEPT and EXELON). The main advantage of these drugs, compared to other known ChEIs, is the reversible or pseudo-reversible binding thereof to the active site of acetylcholine esterase (AChE), which significantly reduces their toxicity.

However, it has been proposed to combine the inhibition of cholinesterase (ChE) with selective protease inhibition function for reducing the formation of β-A4 from APP (1). During the last decade there has been a considerable effort to develop selective muscarinic receptor agonists, such as M1 and M3 receptor agonists. Compounds such as AF102B (2), Lu 25-109 (3), WAY-132983 (4) and Milameline (CI-979/RU35926) (5) have been shown to act as selective M1 (and M3) receptor agonists or partial agonists, while some have been further tested clinically in AD patients (6). In addition, certain nicotinic receptor agonists, which interact with specific subtypes of neuronal nicotinic receptors, have been developed for AD therapy (7).

A combination of two cholinergic functions in the same molecule, such as ChEI with either muscarinic receptor agonist (M1 or M3) or M2 receptor antagonist, has been suggested as well in order to produce higher pharmacological activity (6). The latter has been demonstrated by Amitai et al., who developed novel lipophilic analogs of pyridostigmine (PYR-X) which are both ChEI inhibitors and M2 receptor antagonists (8). Furthermore, BChE and AChE are part of the complex comprising the plaques in AD brain. It was noted by Inestrosa et al. (9) that inhibition by peripheral anionic-site inhibitors decreases the formation of these complexes in AD plaques. Some of these lipophilic PYR-X compounds have been shown to cross the blood-brain barrier (BBB) in rats, and to act as almost equipotent inhibitors of human butyrylcholinesterase (BChE) and acetylcholineesterase (AChE). In this respect it should be noted that the inhibition of brain BChE is as important as AChE inhibition in AD therapy (1, 10).

As mentioned hereinabove, the use of anti-inflammatory drugs is known in the art as well, as a different approach for the treatment of AD. Recent studies have suggested that the symptoms of AD are prevented or attenuated by anti-inflammatory treatment (11). Specifically, non-steroidal anti-inflammatory drugs (NSAIDs) are known to act as inhibitors of the synthesis of IL-6, a cytokine that has been consistently detected in the brains of AD patients, but not in the brains of non-demented elderly persons (12).

The proposed mechanism of the NSAIDs as anti-inflammatory drugs suggests that they inhibit the binding of the prostaglandin substrate, arachidonic acid, to the active site of cyclooxygenase (COX). The constitutive isoform of COX, COX-1, has a clear physiological function in the prostaglandin biosynthesis. The inducible form, COX-2, is formed by pro-inflammatory stimuli in migratory cells and inflamed tissues. Thus, the range of activities of NSAIDs against COX-1 compared to COX-2 influences the variations in the side effects of NSAIDs at their anti-inflammatory effective doses. In other words, the use of NSAIDs for AD treatment is limited by its possible side effects and toxicity. Moreover, most NSAIDs are hydrophilic compounds, and therefore have limited permeability to the brain.

It is therefore a growing need for improved NSAIDs which are more selective COX-2 inhibitors and are further able to cross the BBB, in order to achieve potent anti-inflammatory activity with fewer side effects (13).

Some NSAIDs which are more selective toward COX-2 inhibition are presently known in the art (e.g., IBUPROFEN). However, these NSAIDs hardly cross the BBB, and the use thereof induces side effects, such as gastrointestinal effects. Furthermore, a new generation of NSAIDs, which act as more selective COX-2 inhibitors, has been recently introduced for clinical use [e.g., Searl's Celebrex (celecoxib) and Merck & Co.'s Vioxx (rofecoxib)]. Nevertheless, these new drugs still carry the warning about gastrointestinal effects on their labels, similar to generic NSAIDs (14), and furthermore, it was recently noted that celecoxib may elevate cardiovascular risk in humans (15).

In addition, recent studies showed that both ChEI and anti-inflammatory drugs can be useful for the treatment of acute cerebral ischemia as well (16). In analogy to AD patients, elevated levels of IL-6 have been detected in cerebral spinal fluid (CSF) of patients with acute stroke (17). Furthermore, it has been found that levels of leukotriene LTC4 and prostaglandin E₂ (PGE₂) were higher in cerebral spinal fluid of stroke patients than in age-matched controls (18). Therefore, it is presumed that the use of selective antagonists of LT or inhibitors of its biosynthesis could be helpful in reducing the ischemic penumbra during acute cerebral ischemia, by controlling the vasogenic edema. Furthermore, it was suggested that corticosterone and dexamethasone protection against hypoxic-ischemic damage is a glucocorticoid receptor-mediated effect (19). As is reviewed hereinabove, NSAIDs have been shown to act as inhibitors of prostaglandin biosynthesis, and thus can be useful for treatment of cerebral ischemia and hypoxia.

Moreover, recent studies have demonstrated the use of the ChEI ENA-713 (EXELON) for either protection or treatment of neuronal damage induced by transient brain ischemia in gerbils (20, 21). Post-ischemic administration of EXELON ameliorated the ischemia-induced pyramidal cell loss and reduced significantly the number of glial fibrillary acidic protein-positive astrocytes in the CA1 region of gerbils hippocampus, 14 days post recirculation (21). These findings suggest that ChEIs, such as EXELON, could be useful for treatment of senile dementia such as cerebrovascular dementia, and for reducing the neuronal damage caused by either acute cerebral ischemia or closed head injury.

It is therefore presumed, based on these studies, that the development of new cholinergic compounds such as reversible or irreversible ChEIs, as well as new NSAIDs, can be useful for the treatment and prevention of various CNS disorders and diseases, amongst which are AD, cerebral ischemia, stroke, hypoxia and closed head injury.

Thus, the prior art teaches that cholinergic up-regulators (e.g., ChEIs) and NSAIDs are each independently and via different pharmacotherapy pathways useful in the treatment of CNS disorders and diseases, as well as head injuries and stroke. However, certain cholinergic up-regulators and most NSAIDs, especially those comprising a free carboxylic acid group, are hydrophilic by nature, as they interact with binding sites of enzymes and/or receptors. As such, the brain pharmacopenetration of these compounds is limited.

There is thus a widely recognized need for, and it would be highly advantageous to have novel bifunctional chimeric compounds, covalently coupling a reversible or irreversible cholinergic up-regulator with a NSAID, so as to render the chimera sufficiently hydrophobic so as to freely pass the blood brain barrier, and to exert synergistic copharmacotherapy in the damaged brain.

### SUMMARY OF THE INVENTION

According to the present invention there are provided: (i) chimeric compounds which are conjugates of reversible or irreversible cholinergic up-regulators and non-steroidal anti-inflammatory drugs (NSAIDs); (ii) reversible cholinesterase inhibitors; (iii) methods for their synthesis; and (iv) use thereof for treatment and/or prevention of central nervous system (CNS) disorders and diseases.

It is shown herein that such chimeric compounds have a synergistic effect in several pharmacological aspects including (i) blood brain barrier permeability; (ii) simultaneous and prolonged pharmacokinetics; (iii) colocalized pharmacology; (iv) reversible AChE inhibition; and (v) reduced side effects. Thus, by co-exerting both brain neuronal cholinergic activity, preferably reversible activity, and brain anti-inflammatory activity, the compounds of the present invention have higher pharmacological activity and Therapeutic Index than commonly known ChEIs and prolonged pharmacokinetics as is compared to presently known drugs; they exert preventive (prophylactic) therapy for CNS disorders and diseases; they are sufficiently lipophilic so as to efficiently cross the blood brain barrier; they are pharmacologically active either as a chimeric compound and/or its hydrolytic derivatives; due to the cholinergic up-regulator moiety the compounds of the present invention are targeted (directed) to cholinergic sites where they exert both cholinergic up-regulation and inflammation down-regulation; due to the reversible binding to the cholinergic sites of some of the compounds of the present invention their toxicity is substantially reduced; gastrointestinal side effects, along with other systemic side effects associated with the use of NSAIDs are attenuated due to esterification of the carboxylic acid moiety thereof.

According to one aspect of the present invention there is provided a chimeric compound comprising a cholinergic up-regulator moiety and a non-steroidal anti-inflammatory moiety being covalently linked thereto.

According to another aspect of the present invention there is provided a chimeric compound of a general formula:

A-S-B

wherein:

A is a cholinergic up-regulator moiety selected from the group consisting of a cholinesterase inhibitor residue, a nicotinic receptor agonist residue and a muscarinic receptor agonist residue; B is a non-steroidal anti-inflammatory moiety characterized by a functional group selected from the group consisting of a free carboxylic acid group and a free amine group; and S is a hydrocarbon spacer being covalently linked to B via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to the C atom of the bond via a single covalent bond.

According to yet another aspect of the present invention there is provided a pharmaceutical composition, comprising, as an active ingredient any one or more of the chimeric compounds of the present invention.

According to further features in preferred embodiments of the invention described below, the pharmaceutical composition is formulated for transdermal delivery, nasal administration, administration by inhalation or administration by injection.

According to still another aspect of the present invention there is provided a method of treating, ameliorating or preventing a central nervous system disorder or disease in an organism, the method comprising the step of administering to the organism a therapeutically effective amount of the compound per se or as an active ingredient of a pharmaceutical composition.

According to further features in preferred embodiments of the invention described below, the central nervous system disorder or disease is selected from the group consisting of Alzheimer's disease, cerebrovascular dementia, Parkinson's disease, basal ganglia degenerative diseases, motoneuron diseases, Scrapie, spongyform encephalopathy and Creutzfeldt-Jacob's disease.

According to still further features in the described preferred embodiments the central nervous system disorder or disease is selected from the group consisting of cerebral ischemia, transient hypoxia and stroke.

According to still further features in the described preferred embodiments the central nervous system disorder or disease is a result of a head injury.

According to still further features in the described preferred embodiments the central nervous system disorder or disease is accompanied by an inflammatory process.

According to still further features in the described preferred embodiments the inflammatory process is selected from the group consisting of an inflammatory process induced by infection, an inflammatory process induced by a tumor and an inflammatory process induced by post-operative brain edema.

According to still further features in the described preferred embodiments the infection is selected from the group consisting of viral infection and bacterial infection.

According to still further features in the described preferred embodiments the organism is a mammal.

According to still further features in the described preferred embodiments the mammal is a human being.

According to an additional aspect of the present invention there is provided a method of synthesizing the chimeric compound of the present invention, the method comprising the steps of (a) converting a non-steroidal anti-inflammatory drug into a non-steroidal anti-inflammatory-ester, including a hydrocarbon chain terminating with a reactive halide group; and (b) reacting the non-steroidal anti-inflammatory-ester including the hydrocarbon chain terminating with the reactive halide group with a cholinergic up-regulator, so as to obtain the chimeric compound having the cholinergic up-regulator moiety covalently linked to the non-steroidal anti-inflammatory moiety via the hydrocarbon spacer.

According to yet an additional aspect of the present invention there is provided a method of synthesizing the chimeric compound of the present invention, the method comprising the steps of (a) converting a non-steroidal anti-inflammatory drug into a non-steroidal anti-inflammatory-amide, the amide including a hydrocarbon chain terminating with a reactive halide group; and (b) reacting the non-steroidal anti-inflammatory-amide including the hydrocarbon chain terminating with the reactive halide group with a cholinergic up-regulator, so as to obtain the chimeric compound having said cholinergic up-regulator moiety covalently linked to said non-steroidal anti-inflammatory moiety via said hydrocarbon spacer.

According to still an additional aspect of the present invention there is provided a method of synthesizing the chimeric compound of the present invention, the method comprising the steps of (a) converting a cholinergic up-regulator into its N(ring)-substituted derivative, the derivative including a hydrocarbon chain terminating with a reactive hydroxyl group; and (b) reacting the N(ring)-substituted derivative including the hydrocarbon chain terminating with the reactive hydroxyl group with a reactive derivative of a non-steroidal anti-inflammatory drug, so as to obtain the chimeric compound having the cholinergic up-regulator moiety covalently linked to the non-steroidal anti-inflammatory moiety. Optionally, the method further comprising the step of converting the N(ring)-substituted derivative including the hydrocarbon chain terminating with the reactive hydroxyl group into a tertiary amine N(ring)-substituted derivative including the hydrocarbon chain terminating with the reactive hydroxyl group, prior to the step (b).

According to further features in preferred embodiments of the invention described below, the cholinergic up-regulator moiety and the non-steroidal anti-inflammatory moiety are covalently linked via a hydrocarbon spacer.

According to still further features in the described preferred embodiments the non-steroidal anti-inflammatory moiety is covalently attached to the spacer via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to the C atom of the bond via a single covalent bond.

According to still further features in the described preferred embodiments the —C(═X)Y— bond is selected from the group consisting of an ester bond and an amide bond.

According to still further features in the described preferred embodiments the ester bond is selected from the group consisting of a carboxylic ester bond and a glycol amide ester bond.

According to still further features in the described preferred embodiments the —C(═X)Y— bond is hydrolizable by brain derived esterases or amidases.

According to still further features in the described preferred embodiments the hydrocarbon spacer comprises at least one hydrocarbon selected from the group consisting of an alkyl having 2-20 carbon atoms, a cycloalkyl having 3-20 carbon atoms and an aryl having 6-20 carbon atoms.

According to still further features in the described preferred embodiments the cholinergic up-regulator moiety is selected from the group consisting of a reversible cholinesterase inhibitor and an irreversible cholinesterase inhibitor.

According to still further features in the described preferred embodiments the cholinergic up-regulator moiety is selected from the group consisting of a cholinesterase inhibitor residue, a nicotinic receptor agonist residue and a muscarinic receptor agonist residue.

According to still further features in the described preferred embodiments the cholinesterase inhibitor residue is a pyridostigmine residue.

According to still further features in the described preferred embodiments the pyridostigmine residue is a 3-N,N-dimethylcarbamoyl pyridinium bromide residue.

According to still further features in the described preferred embodiments the nicotinic agonist residue is selected from the group consisting of a nicotine residue and a cytisine residue.

According to still further features in the described preferred embodiments the muscarinic receptor agonist residue is selected from the group consisting of an arecoline residue and a pilocarpine residue.

According to still further features in the described preferred embodiments the non-steroidal anti-inflammatory moiety comprises a residue of a non-steroidal anti-inflammatory drug characterized by a functional group selected from the group consisting of a free carboxylic acid group and a free amine group.

According to still further features in the described preferred embodiments the non-steroidal anti-inflammatory moiety is selected from the group consisting of an ibuprofen residue, an indomethacin residue, a naproxen residue, a diclofenac residue and an aspirin residue.

According to still further features in the described preferred embodiments the ibuprofen residue is selected from the group consisting of an (±)-ibuprofen residue, an S-(+)-ibuprofen residue and an R-(−)-ibuprofen residue.

According to still further features in the described preferred embodiments the chimeric compound is characterized by lipophilicity sufficient for permitting the compound to cross a blood brain barrier of an organism.

According to still further features in the described preferred embodiments the chimeric compound is characterized by cholinergic up-regulation activity and inflammation down-regulation activity exerted by the chimeric compound and/or hydrolytic derivatives thereof, and hence may be defined as a drug and/or a prodrug.

According to a further aspect of the present invention there is provided a reversible cholinesterase inhibitor having a general formula A:

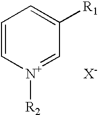

wherein:

R₁ is C(═Q)-Z-R₃; R₂ is selected from the group consisting of hydrogen, an alkyl, a hydroxyalkyl, a haloalkyl, an alkylamine, a cycloalkyl and an aryl; X is a halide; Q and Z are each independently selected from the group consisting of oxygen and sulfur; and R₃ is selected from the group consisting of an alkyl, a cycloalkyl and an aryl.

According to yet a further aspect of the present invention there is provided a method of synthesizing the reversible cholinesterase inhibitor, the method comprising reacting a pyridine ring substituted at position 3 by the R₁ with a R₂ residue terminating with the halide group X, so as to produce a quaternary pyridinium halide substituted at the N(ring) position by the R₂ residue and at position 3 by the R₁ residue.

According to further features in preferred embodiments of the invention described below, Q and Z are each oxygen, R₃ is methyl, R₂ is an alkyl and X is selected from the group consisting of bromide and iodide.

According to still a further aspect of the present invention there is provided a reversible cholinesterase inhibitor having a general formula B:

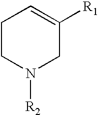

wherein:

R₁ is C(═Q)-Z-R₃; R₂ is selected from the group consisting of hydrogen, an alkyl, a hydroxyalkyl, a haloalkyl, an alkylamine, a cycloalkyl and an aryl; Q and Z are each independently selected from the group consisting of oxygen and sulfur; and R₃ is selected from the group consisting of an alkyl, a cycloalkyl and an aryl.

According to another aspect of the present invention, there is provided a method of synthesizing the reversible cholinesterase inhibitor, comprising: (a) reacting a pyridine ring substituted at position 3 by the R₁ with an organic halide and/or a reactive inorganic halide, so as to produce a quaternary pyridinium halide substituted by the R₁ at position 3; and (b) reducing the quaternary pyridinium halide, so as to produce a tertiary tetrahydropyridine substituted by the R₁ at position 3.

According to further features in preferred embodiments of the invention described below, Q and Z are each oxygen, R₃ is methyl and R₂ is an alkyl.

According to still further features in the described preferred embodiments the reactive inorganic halide is potassium iodide.

According to still further features in the described preferred embodiments the organic halide is the R₂ residue terminating with the halide group X and the quaternary pyridinium halide is further substituted at the N(ring) position by the R₂ residue.

According to yet another aspect of the present invention there is provided a method of treating, ameliorating or preventing a central nervous system disorder or disease, as described hereinabove, in an organism, the method comprising the step of administering to the organism a therapeutically effective amount of the reversible cholinesterase inhibitor per se or as an active ingredient of a pharmaceutical composition.

The present invention successfully addresses the shortcomings of the presently known configurations by providing new and potent chimeric compounds for the treatment and prevention of central nervous system disorders and diseases.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are plots demonstrating a time-course of HuAChE (FIG. 1a) and FBS-AChE (FIG. 1b) inhibition in vitro by varying concentrations of DICLO-PO (at 25° C., in phosphate buffer, 50 Mm, pH 7.4);

FIG. 2 is a plot demonstrating a time-course of whole blood ChE activity following intramuscular administration of PYR, PO and IBU-PO in mice;

FIG. 3 shows photographs of rat stomach mucosal tissue following intraperitoneal administration of 10 mg/kg IBU-PO and DICLO-PO, note the absence of erosions or ulcers;

FIG. 4 is a bar graph demonstrating a decrease in carrageenan-induced rat paw edema following intraperitoneal injection of 5 mg/kg NSAIDs and NSAID-PYR-X compounds, compared with injection of a vehicle only;

FIG. 5 is a comparative bar graph demonstrating an effect of IBU and IBU-PO on carrageenan-induced brain edema in rats;

FIG. 6 is a bar graph demonstrating the effect of intraperitoneal injection of 10 mg/kg IBU-PO on carrageenan-induced brain edema in mice;

FIG. 7 shows comparative plots demonstrating the effect of pretreatment with 5 mg/kg atropine and 2 mg/kg mecamylamine on IBU-PO-induced (by intraperitoneal injection of 2.5 mg/kg IBU-PO) hypothermia in mice;

FIG. 8 is a bar graph demonstrating the effect of varying doses of PO, IBU-PO and NAPRO-PO on edema level induced by a closed head injury in mice;

FIG. 9 is a bar graph demonstrating the effect of treatment with varying doses of IBU-PO on survival time in male mice following hypobaric hypoxia [n=4 per group], compared with a control group treated with a vehicle only;

FIG. 10 is a bar graph demonstrating the effect of IBU-PO and known ChE inhibitors on survival time in mice during hypobaric hypoxia [n=4 per group];

FIG. 11 shows plots demonstrating the competition binding curves for the displacement of [³H]NMS from rat brain by NSAID-PYR-X compounds; and

FIG. 12 shows plots demonstrating the competition binding curves for IBU-PO with various radioactive ligands in rat brain.

### DESCRIPTION OF THE PREFERRED EMBODIMENTS

The present invention is of chimeric compounds which are conjugates of reversible or irreversible cholinergic up-regulators and non-steroidal anti-inflammatory drugs (NSAIDs) and of novel reversible cholinesterase inhibitors, pharmaceutical compositions containing same, methods of their preparation and their use for the treatment of central nervous system (CNS) disorders and diseases, such as, but not limited to, Alzheimer's disease, cerebrovascular dementia, cerebral ischemia, transient hypoxia and stroke, as well as CNS diseases or disorders induced by closed head injury or accompanied by inflammatory processes.

The principles and operation of the chimeric compounds according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While conceiving the present invention, it was hypothesized that a chimeric compound covalently coupling a cholinergic up-regulator moiety and a NSAID moiety could exert synergistic pharmacological activity toward CNS disorders and diseases. The underlying concepts of this hypothesis are as follows:

CNS disorders and diseases are in many cases characterized by reduced cholinergic activity and inflammation. It is indeed known for many years that CNS disorders and diseases are treatable via hydrophobic derivatives of cholinergic up-regulators and potentially treatable by anti-inflammatory drugs. However, certain cholinergic up-regulators and some non-steroidal anti-inflammatory drugs, especially those comprising a free carboxylic acid group, are hydrophilic and therefore fail to efficiently cross the blood brain barrier, where they are to exert their therapeutic activity.

It was, therefore, hypothesized that covalently coupling a cholinergic up-regulator and a non-steroidal anti-inflammatory drug via a bond hydrolizable by brain enzymes, one would achieve a synergistic effect in several aspects including (i) blood brain barrier permeability; (ii) simultaneous and prolonged pharmacokinetics; (iii) co-localized pharmacology; (iv) reduced side effects.

While reducing the present invention to practice, as is further exemplified in the Examples section that follows, it was found that covalently coupling a cholinergic up-regulator and a non-steroidal anti-inflammatory drug via a bond hydrolizable by brain enzymes results in (i) a combined action of cholinergic up-regulation and inflammation down regulation of both the chimeric compound and its hydrolytic derivatives, characterized by unitary pharmacokinetics; (ii) sufficient lipophilicity to cross the BBB; (iii) high affinity to brain cholinergic receptors; (iv) lower toxicity; (v) larger Therapeutic Index; and (vi) longer duration of action in the brain compared with other known drugs used for treating CNS disorders and diseases.

Thus, the chimeric compounds are used according to the present invention to treat CNS disorders and diseases. Each of the compounds which are used to treat CNS disorders and diseases according to the present invention includes a cholinergic up-regulator moiety and a non-steroidal anti-inflammatory moiety which are covalently coupled.

As used herein in the specification and in the claims section that follows, the term “cholinergic up-regulator moiety” refers to a residue derived from a cholinergic compound which retains its cholinergic activity. As is well accepted in the art, the term “residue” refers herein to a major portion of a molecule which is covalently linked to another molecule.

Acetylcholine (ACh) is a neurotransmitter that is constantly produced by a synthetic pathway involving choline acetyltransferase (ChAT), decomposed by cholinesterase (ChE) and exerts its cholinergic function via acetylcholine receptors. Thus, cholinergic up regulation can be achieved by (i) activating its synthetic pathway; (ii) blocking its degradation by inhibition of ChEs; and/or (iii) mimicking its action via agonists directed towards cholinergic receptors.

Thus, cholinergic compounds according to the present invention include, for example, cholinesterase inhibitors (ChEI) such as, but not limited to, a pyridostigmine; nicotinic receptor agonists such as, but not limited to, nicotine and cytisine, or muscarinic receptor agonists such as, but not limited to, arecoline and pilocarpine.

The term “non-steroidal anti-inflammatory (NSAID) moiety” as used herein refers to a residue, as this term is defined hereinabove, of a non-steroidal anti-inflammatory drug characterized by a functional group such as, but not limited to, a free carboxylic acid group or a free amine group. NSAIDs according to the present invention include, for example, ibuprofen, indomethacin, naproxen, diclofenac and aspirin.

According to a preferred embodiment of the present invention, the non-steroidal anti-inflammatory moiety is an (±)-ibuprofen residue, an S-(+)-ibuprofen residue or an R-(−)-ibuprofen residue. The pure optical isomer S-(+)-ibuprofen, also referred to in the art as dexibuprofen, is known to exert enhanced anti-inflammatory activity compared to the ibuprofen racemic mixture and is thus used as an efficacious drug for rheumatoid arthritis and osteoarthritis (35, 36). However, the other optical isomer, R-(−)-ibuprofen, which is known to act as an effective anti-inflammatory drug as well, has been recently found to further act as an anticancer drug (37). It was therefore expected that a chimeric compound comprising an S-(+)-ibuprofen residue or an R-(−)-ibuprofen residue will exert higher pharmacological activity. Indeed, as is shown hereinbelow in the Examples section, the chimeric compound obtained from the optical isomer S-(+)-ibuprofen was found to exert a pharmacological activity that is about 10 times higher than the chimeric compound obtained from the racemic ibuprofen.

According to another preferred embodiment of the present invention, the cholinergic up-regulator moiety and the NSAID moiety are covalently linked via a hydrocarbon spacer.

As used herein in the specification and in the claims section that follows, the term “hydrocarbon” refers to a compound that includes hydrogen atoms and carbon atoms which are covalently attached. The hydrocarbon can be saturated, unsaturated, branched or unbranched.

The term “hydrocarbon spacer” refers to a hydrocarbon moiety comprised of at least one hydrocarbon, such as, but not limited to, alkyl, cycloalkyl and/or aryl.

As used herein in the specification and in the claims section that follows, the term “alkyl” refers to a saturated aliphatic hydrocarbon including straight chain and branched chain groups. Preferably, the alkyl group has 2 to 20 carbon atoms. Whenever a numerical range; e.g., “2-20”, is used herein, it means that the group, in this case the alkyl group, may contain 2 carbon atoms, 3 carbon atoms, etc., up to and including 20 carbon atoms. More preferably, it is a medium size alkyl having 4 to 16 carbon atoms. Most preferably, it is an alkyl having 8 to 14 carbon atoms.

A “cycloalkyl” group refers to an all-carbon monocyclic or fused ring groups (i.e., rings which share an adjacent pair of carbon atoms), wherein one or more of the rings does not have a completely conjugated pi-electron system. Examples, without limitation, of cycloalkyl groups are cyclopropane, cyclobutane, cyclopentane, cyclohexane, cyclohexadiene, cycloheptane, cycloheptatriene and adamantane.

An “aryl” group refers to an all-carbon monocyclic or fused-ring polycyclic group (i.e., rings which share adjacent pairs of carbon atoms) having a completely conjugated pi-electron system. Examples, without limitation, of aryl groups are phenyl, naphthalenyl and anthracenyl.

According to a preferred embodiment of the present invention, the NSAID moiety is covalently attached to the hydrocarbon spacer via a “—C(═X)Y—” bond, where X is, without limitation, a non-substituted or substituted oxygen, sulfur or nitrogen atom and Y is, without limitation, a substituted or non-substituted carbon, oxygen, nitrogen, sulfur, silicon or phosphor atom linked to the C atom of the bond via a single covalent bond.

According to another preferred embodiment of the present invention, the “—C(═X)Y—” bond is an ester bond or an amide bond.

As used herein in the specification and in the claims section that follows, the term “ester bond” refers to a “—C(═X)Y—” bond, where X is without limitation, oxygen or sulfur, and Y is, without limitation, oxygen, sulfur, glycol amine, glycol ester, O-carbamyl, or O-thiocarbamyl.

A “glycol amine” group refers to an —O—CH₂—C(═O)—NR′— group, where R′ is hydrogen, alkyl, cycloalkyl or aryl.

A “glycol ester” group refers to an —O—CH₂—C(═O)—OR′— group, where R′ is as defined above.

An “O-carbamyl” group refers to an —O—C(═O)—NR′— group, where R′ is as defined above.

An “O-thiocarbamyl” group refers to an —O—C(═S)—NR′— group, where R′ is as defined above.

The term “amide bond” as used herein refers to a “—C(═X)Y—” bond, where X is, without limitation, oxygen or sulfur, and Y is, without limitation, amine, N-carbamyl or N-thiocarbamyl.

An “amine” group refers to a —NR′— group, where R′ is hydrogen, alkyl, cycloalkyl or aryl.

A “N-carbamyl” group refers to a —NR′—C(═O)—O-R′— group, where R′ is as defined above.

A “N-thiocarbamyl” group refers to a —NR′—C(═S)—O—R′— group, where R′ is as defined above.

According to a preferred embodiment of the present invention, the ester bond is a carboxylic ester bond or a glycol amide ester bond.

As used herein in the specification and in the claims section that follows, the term “carboxylic ester bond” refers to a “—C(═O)O—” bond.

The term “glycol amide ester bond” as used herein refers to a “C(═O)O—CH₂—C(═O)—NR“—” bond, where R″ is hydrogen or alkyl.

According to another preferred embodiment of the present invention, the “—C(═Y)X—” bond is hydrolizable by one or more brain derived esterases or amidases. Such hydrolysis provides for slow release of the NSAID moiety from the chimeric compound in brain, and its rate is determined by the nature of the ester bond employed (22). The outcome of such controlled release of the NSAID moiety is a longer duration of action for the compound in the brain, following one administration.

However, according to another preferred embodiment of the present invention, the chimeric compound is characterized by cholinergic up-regulation activity and inflammation down-regulation activity exerted by the chimeric compound and/or its hydrolytic derivatives.

The term “hydrolytic derivatives”, according to the present invention, refers to the products formed in vivo by the enzymatic hydrolysis described hereinabove.

Thus, the pharmacological activity of the chimeric compound of the present invention is exerted by both a prodrug, which is the parent chimeric compound itself, and by one or more drugs derived therefrom, which are the hydrolytic derivatives of the chimeric compound. This dual activity of both a prodrug and drugs derived therefrom is a novel pharmacological concept.

According to a preferred embodiment of the present invention, the chimeric compound is a NSAID-PYR-X compound whose chemical structure is described in Scheme I below:

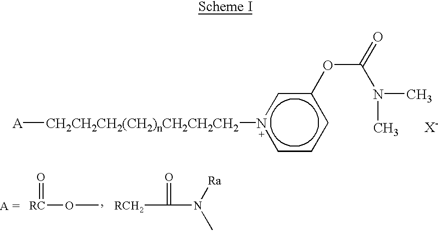

where: n=2, 4, 6; Ra=H or alkyl and X=halide

where R is one of:

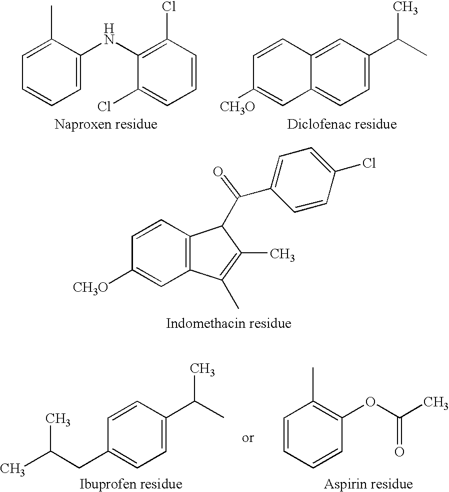

As used herein in the specification and in the claims section that follows, the term “NSAID-PYR-X” refers to a chimeric compound comprised of a 3-N,N-dimethylcarbamoyl pyridinium bromide residue which is covalently linked through an ester bond or an amide bond to a NSAID residue, via a hydrocarbon spacer.

The term “3-N,N-dimethylcarbamoyl pyridinium bromide” as used herein refers to a pyridinium bromide moiety substituted at position 3 thereof with an —O—C(═O)—N(CH₃)₂— group.

According to another preferred embodiment of the present invention, the chimeric compound is an IBU-4H-PO compound whose chemical structure is described in Scheme II below:

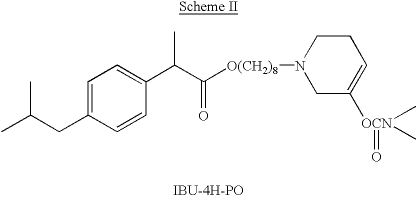

As used herein in the specification and in the claims section that follows, the term “IBU-4H-PO” refers to 2-(4-isobutyl phenyl)-propionic acid 8-(3-N,N-dimethylcarbaboyl-3,6-dihydro-2H-pyridine-1-yl)-octyl ester.

According to still another preferred embodiment of the present invention, the chimeric compound is an IBU-OCT-cytisine compound (nicotinic receptor agonist) whose chemical structure is described in Scheme III below:

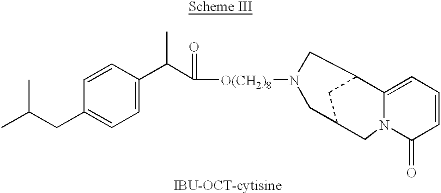

The term “IBU-OCT-cytisine” as used herein refers to ibuprofen N-octyl-cytisine ester.

Reversible cholinesterase inhibitors:

While continuing to explore the chimeric compounds of the present invention, the compounds IBU-OCT-arecoline (a muscarinic receptor agonist), also referred to hereinafter as IOA, and IBU-OCT-methylnicotinate, also referred to hereinafter as IOMN, were prepared. Their chemical structures are described in Scheme IV below:

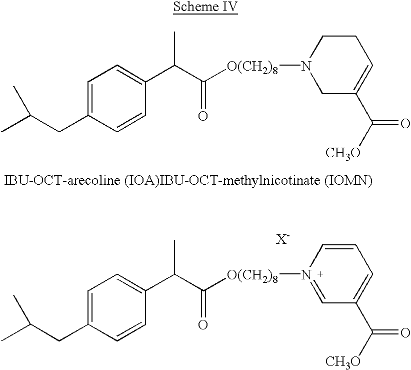

As used herein in the specification and in the claims section that follows, the term “IBU-OCT-arecoline” (IOA) refers to 1-{8-[2-(4-isobutyl-phenyl)-propionyl]-octyl}- 1,2,5,6-tetrahydropyridine-3-carboxylic acid methyl ester.

The term “IBU-OCT-methyl nicotinate” (IOMN) refers to 1-{8-[2-(4-isobutyl-phenyl)-propionyl]-octyl}-3-methoxycarbonyl pyridinium iodide.

However, while evaluating the pharmacological activity of the IOA and IOMN compounds, it was surprisingly found, as is further described and exemplified in the Examples section, that these compounds act as reversible cholinesterase inhibitors.

Thus, contrary to the NSAID-PYR-X compounds of the present invention and other known ChEIs, which inhibit the cholinesterase by covalently (and therefore irreversibly) carbamylating the serine residue at the active site of the enzyme, the IOMN and IOA compounds interact with the enzyme via electrostatic and hydrophobic interactions which are completely reversible. This reversible inhibition activity is highly advantageous since it substantially reduces the toxicity of the compounds. At this point, it is pertinent to note that the presently known Alzheimer's disease drugs which are approved by the FDA are ARICEPT and EXELON, which are reversible and pseudo-reversible AChE inhibitors, respectively.

In a search for new reversible AChE inhibitors, it was found that the known compounds arecoline and methyl nicotinate (MN) are by themselves reversible AChE inhibitors.

Thus, according to another aspect of the present invention there is provided a reversible cholinesterase inhibitor having a general formula A:

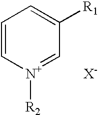

wherein:

R₁ is C(═Q)-Z-R₃; R₂ is selected from the group consisting of hydrogen, an alkyl, a hydroxyalkyl, a haloalkyl, an alkylamine, a cycloalkyl and an aryl; X is a halide; Q and Z are each independently selected from the group consisting of oxygen and sulfur; and R₃ is selected from the group consisting of an alkyl, a cycloalkyl and an aryl.

Further according to the present invention, there is provided another reversible cholinesterase inhibitor having a general formula B:

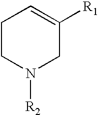

wherein:

R₁ is C(═Q)-Z-R₃; R₂ is selected from the group consisting of hydrogen, an alkyl, a hydroxyalkyl, a haloalkyl, an alkylamine, a cycloalkyl and an aryl; Q and Z are each independently selected from the group consisting of oxygen and sulfur; and R₃ is selected from the group consisting of an alkyl, a cycloalkyl and an aryl.

The term “haloalkyl”, as used herein in the specification and in the claims section that follows, refers to an alkyl group as defined hereinabove, which include at least one carbon atom that is substituted by a halogen.

The term “hydroxyalkyl” as used herein, refers to an alkyl group as defined hereinabove, which includes at least one carbon atom that is substituted by an —OH group.

The term “alkylamine” as used herein, refers to an alkyl group as defined hereinabove, which includes at least one carbon atom that is substituted by an amine group as defined hereinabove.

Chemical synthesis:

Further according to the present invention, there are provided methods for synthesizing the chimeric compounds of the present invention.

A first method according to the present invention is effected by converting a non-steroidal anti-inflammatory drug into a non-steroidal anti-inflammatory-ester which includes a hydrocarbon chain terminating with a reactive halide group, and thereafter reacting the non-steroidal anti-inflammatory-ester with a cholinergic up-regulator, thereby obtaining a chimeric compound having a cholinergic up-regulator moiety and a non-steroidal anti-inflammatory moiety being covalently linked thereto via a hydrocarbon spacer and an ester bond.

The term “derivative” as used herein refers to the result of a chemically altering, modifying or changing a molecule or a portion thereof, such that it maintains its original functionality in at least one respect.

The reactive halide group can be fluoride, chloride, bromide, or iodide.

In one particular, the non-steroidal anti-inflammatory drug which includes a free carboxylic acid group is converted into its acetyl chloride derivative via interaction with an active nucleophilic chloride, such as oxalyl chloride. Then, the anti-inflammatory drug acetyl chloride derivative is esterified by a hydrocarbon terminated at a first end thereof with a hydroxyl and at the opposing end thereof with a halide such as bromide. Then, the esterified anti-inflammatory drug is reacted with a cholinergic up-regulator which includes a pyridine ring to form a chimeric compound having a cholinergic up-regulator moiety and a non-steroidal anti-inflammatory moiety being covalently linked thereto via a hydrocarbon spacer and a carboxylic ester bond and being characterized by a quaternary ammonium halide residue.

A second method according to the present invention is effected by converting a non-steroidal anti-inflammatory drug into a non-steroidal anti-inflammatory-amide which includes a hydrocarbon chain terminating with a reactive halide group, and thereafter reacting the non-steroidal anti-inflammatory-amide with a cholinergic up-regulator, thereby obtaining a chimeric compound having a cholinergic up-regulator moiety and a non-steroidal anti-inflammatory moiety being covalently linked thereto via a hydrocarbon spacer and an amide bond.

In one particular, the non-steroidal anti-inflammatory drug which includes a free carboxylic acid group is converted into its acetyl chloride derivative via interaction with an active nucleophilic chloride, such as oxalyl chloride. Then, the anti-inflammatory drug acetyl chloride derivative is reacted with a hydrocarbon terminated at a first end thereof with an amine and at the opposing end thereof with a halide such as bromide to form an anti-inflammatory drug amide derivative. Then, the anti-inflammatory drug amide derivative is reacted with a cholinergic up-regulator which includes a pyridine ring to form a chimeric compound having a cholinergic up-regulator moiety and a non-steroidal anti-inflammatory moiety being covalently linked thereto via a hydrocarbon spacer and an amide bond and being characterized by a quaternary ammonium halide residue.

A third method according to the present invention is effected by converting a cholinergic up-regulator into its N(ring)-substituted derivative, where the derivative includes a hydrocarbon chain terminating with a reactive hydroxyl group, and thereafter reacting the N(ring)-substituted derivative with a derivative of a non-steroidal anti-inflammatory drug. Optionally, this method further includes the step of converting the N(ring)-substituted derivative into its tertiary amine N(ring)-substituted derivative, prior to the reaction with the derivative of the non-steroidal anti-inflammatory drug.

In one particular, a cholinergic up-regulator which includes a ring moiety that contains a nitrogen atom, such as pyridine or cytisine, is reacted with a hydrocarbon terminated at a first end thereof with a hydroxyl and at the opposing end thereof with a halide such as bromide, to form a quaternary ammonium halide derivative of the cholinergic up-regulator, substituted at its N-ring with a hydrocarbon terminated with a hydroxyl. Then, an acetyl chloride derivative of a non-steroidal anti-inflammatory drug is esterified by the hydroxyl of the quaternary ammonium derivative, to form a chimeric compound having a cholinergic up-regulator moiety and a non-steroidal anti-inflammatory moiety being covalently linked thereto via a hydrocarbon spacer and an ester bond, and being characterized by a quaternary ammonium halide residue.

In another particular, the quaternary ammonium derivative of the cholinergic up-regulator, which is substituted at its N-ring with a hydrocarbon terminated with a hydroxyl, is reduced into a tertiary amine derivative. The acetyl chloride derivative of a non-steroidal anti-inflammatory drug is then esterified by the hydroxyl of the tertiary amine derivative, to form a chimeric compound having a cholinergic up-regulator moiety and a non-steroidal anti-inflammatory moiety being covalently linked thereto via a hydrocarbon spacer and an ester bond and being characterized by a reduced tertiary amine residue.

Further according to the present invention, there are provided methods of synthesizing the reversible cholinesterase inhibitors of the present invention.

A first method according to the present invention is effected by reacting a pyridine ring that is substituted at position 3 by a carboxylate group with a substituted or non-substituted residue terminating with a halide group, to form a quaternary pyridinium ring substituted by the substituted or non-substituted residue at the N(ring) position and by the carboxylate group at position 3.

The term “carboxylate group” as used herein refers to a —C(═Q)Z-R′″-”group, where Q and Z are each independently oxygen or sulfur and R′″ is, without limitation, alkyl, cycloalkyl or aryl, as defined hereinabove. Representative examples of a carboxylate group are methyl acetate, methyl thioacetate and ethyl acetate.

Representative examples of a substituted or non-substituted residue are alkyl, cycloalkyl, haloalkyl, hydroxyalkyl, alkylamine and aryl, as defined hereinabove.

Representative examples of a halide group are bromide and iodide.

Thus, in one particular, a pyridine ring that is substituted at position 3 by a carboxylate group, such as methyl nicotinate, is reacted with a substituted or non-substituted alkyl terminating with a halide, such as bromide or iodide, to form a quaternary pyridinium ring that is substituted by a carboxylate group at position 3 and by the substituted or non-substituted alkyl at the N(ring) position.

A second method according to the present invention is effected by reacting a pyridine ring that is substituted at position 3 by a carboxylate group with an organic halide and/or a reactive inorganic halide, to form a quaternary pyridinium halide that is substituted at position 3 by carboxylate group, and reducing thereafter the formed quaternary pyridinium halide, to form a tertiary tetrahydropyridine ring that is substituted at position 3 by a carboxylate group.

Representative examples of a reactive inorganic halide are potassium fluoride, potassium iodide, sodium iodide and sodium bromide.

The term “organic halide” as used herein refers to a substituted or non-substituted residue, as defined hereinabove, that includes a halide group at its end.

In one particular, a pyridine ring that is substituted at position 3 by a carboxylate group, such as methyl nicotinate, is reacted with a substituted or non-substituted alkyl terminating with a halide group, such as bromide or iodide, and with a reactive inorganic halide, such as potassium iodide, to form a quaternary pyridinium ring that is substituted by a carboxylate at position 3 and by the substituted or non-substituted alkyl at the N(ring) position. The substituted quaternary pyridinium ring is then reduced, to form a tertiary tetrahydropyridine ring that is substituted by a carboxylate group at position 3 and by the substituted or non-substituted alkyl at the N(ring) position.

Pharmaceutical composition:

Further according to the present invention there is provided a pharmaceutical composition including the chimeric compound of the invention as an active ingredient.

As used herein a “pharmaceutical composition” refers to a preparation of one or more of the chimeric compounds described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water. Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Routes of administration: Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Composition/formulation: Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer with or without organic solvents such as propylene glycol, polyethylene glycol. For transmucosal administration, penetrants are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The chimeric compounds described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile, pyrogen-free water, before use.

The chimeric compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Dosage: Pharmaceutical compositions suitable for use in context of the present invention include compositions wherein the active ingredients are contained in an amount effective to achieve the intended purpose. More specifically, a therapeutically effective amount means an amount of chimeric compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any chimeric compound used in the methods of the invention, the therapeutically effective amount or dose can be estimated initially from activity assays in animals. For example, a dose can be formulated in animal models to achieve a circulating concentration range that includes the IC₅₀ as determined by activity assays (e.g., the concentration of the test compound, which achieves a half-maximal inhibition of the ChE or COX activity). Such information can be used to more accurately determine useful doses in humans.

Toxicity and therapeutic efficacy of the compounds described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active moiety which are sufficient to maintain the cholinesterase (ChE) modulating effects, termed the minimal effective concentration (MEC). The MEC will vary for each preparation, but can be estimated from in vitro data; e.g., the concentration necessary to achieve 50-90% inhibition of a ChE may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Packaging: Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a chimeric compound of the invention formulated in a compatible pharmaceutical carrier may also be prepared, placed in an appropriate container, and labeled for treatment of an indicated condition. Suitable conditions indicated on the label may include treatment of an Alzheimer's disease, cerebral ischemia, stroke and a closed head injury.

Pharmacology:

Further according to the present invention, there is provided a method for treating, ameliorating or preventing a central nervous system disorder or disease in an organism (e.g., a human being). The method is effected by administering a therapeutically effective amount of one or more of the chimeric compounds of the invention to a treated subject.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease, substantially ameliorating clinical symptoms of a disease or substantially preventing the appearance of clinical symptoms of a disease.

As used herein, the term “CNS disorder or disease” refers to a disorder or disease characterized by an impairment of the CNS. The impairment can be affected by both down-regulation of acetylcholine and an inflammation process.

The term “administering” as used herein refers to a method for bringing a chimeric compound of the present invention into an area or a site in the brain that is impaired by the CNS disorder or disease.

The term “organism” refers to animals, typically mammals having a blood brain barrier, including human.

The term “therapeutically effective amount” refers to that amount of the compound being administered which will relieve to some extent one or more of the symptoms of the disorder or disease being treated.

The present invention is thus directed to chimeric compounds which are capable of crossing the blood brain barrier, so as to approach the impaired site or area in the brain, and cause cholinergic up-regulation together with down-regulation of the inflammation process.

Examples of diseases associated with CNS impairment that are treatable using the chimeric compounds of the invention, include, without limitation, Alzheimer's disease, cerebrovascular dementia, Parkinson's disease, basal ganglia degenerative diseases, motoneuron diseases, Scrapie, spongyform encephalopathy and Creutzfeldt-Jacob's disease,

Examples of disorders associated with CNS impairment that are treatable using the chimeric compounds of the invention, include, without limitation, cerebral ischemia, transient hypoxia, and stroke. Further disorders can be induced by closed head injury, infection, tumor and post-operative brain edema.

Further according to the present invention, there is provided a method for treating, ameliorating or preventing a central nervous system disorder or disease in an organism (e.g., a human being). The method is effected by administering a therapeutically effective amount of one or more of the reversible cholinesterase inhibitors of the invention to a treated subject, either per se or as an active ingredient in a pharmaceutical composition.

The reversible cholinesterase inhibitors of the invention cause reversible cholinergic up-regulation at the impaired site or area in the brain, and may therefore be used in association with CNS disorders and diseases as defined hereinabove.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### MATERIALS AND EXPERIMENTAL METHODS

### Chemical Syntheses and Analyses

The following procedures describe the syntheses and analyses of the bifunctional chimeric compounds of the invention, and the intermediates thereof.

Synthesis of indomethacin acid chloride: Indomethacin (2.3 grams, 6.4 mmol) was placed in a dried, nitrogen-purged, 3-necked 100 ml round bottom flask. Oxalyl chloride (5.7 grams, 45 mmol) was then added dropwise, and the reaction was allowed to proceed at room temperature until evolution of gases ceased. Evaporation (herein and below, under reduced pressure) of unreacted oxalyl chloride resulted in the formation of the product as a pale yellow solid (1.6 grams, 66% yield).

¹H-NMR (CDCl₃): δ=2.41 (s, CH₃, 3H), 3.81 (s, OCH₃, 3H), 4.17 (s, CH₂C(O), 2H), 6.6 (d, Indo-H₇), 6.85 (d, indo-H₆), 6.9 (s, indo-H₄), 7.46 (d, Hα, 2H), 7.6 (d, Hβ, 2H) ppm.

Synthesis of indomethacin bromooctyl ester: Indomethacin acid chloride (1.6 grams, 4.25 mmol) and 1-bromo-8-octanol (0.83 gram, 4 mmol), in dichloromethane (50 ml), were refluxed for 3 days. Evaporation of the solvent, followed by purification of the resulting crude oil by silica gel column chromatography, using a mixture of 70%/30% ether/hexane, gave 1.2 grams of the product (50.5% yield).

¹H-NMR (CDCl₃): δ1.27 (m, CH₂, 8H), 1.61 (quin, CH₂CH₂Br, 2H), 1.82 (quin, OCH₂CH₂, 2H), 2.38 (s, CH₃, 3H), 3.39 (t, CH₂Br, 2H), 3.65 (s, CH₂C(O), 2H), 3.83 (s, OCH₃, 3H), 4.1 (s, CH₂O, 2H), 6.6 (d, indo-H₇), 6.85 (d, indo-H₆), 6.9 (s, indo-H₄), 7.46 (d, Ha, 2H), 7.6 (d, Hβ, 2H) ppm.

Synthesis of N-(Indomethacin octanoate)-3-N,N-dimethylcarbamoyl pyridinium bromide (INDO-PO): Indomethacin bromooctyl ester (1 gram, 1.8 mmol) and 3-dimethylcarbamoyl pyridine (0.7 gram, 4.2 mmol), in methoxy ethanol (50 ml), were refluxed for 2 days. Concentration (herein and below, under vacuum) and purification of the resulting crude oil by silica gel column chromatography, using a mixture of 17:3 chloroform/methanol, gave 0.53 gram product (41% yield).

¹H-NMR (CDCl₃): δ=1.25 (m, CH₂, 8H), 1.58 (quin, OCH₂CH₂, 2H), 2.01 (quin, CH₂CH₂N, 2H), 2.36 (s, CH₃, 3H), 3.04 (s, NCH₃, 3H), 3.16 (s, NCH₃, 3H), 3.65 (s, CH₂C(O), 2H), 3.82 (s, OCH₃, 3H), 4.1 (s, CH₂O, 2H), 4.92 (t, CH₂N, 2H), 6.6 (d, indo-H₇), 6.85 (d, indo-H₆), 6.9 (s, indo-H₄), 7.46 (d, Hα, 2H), 7.6 (d, Hβ, 2H), 8.1 (t, pyr-H₅), 8.26 (d, pyr-H₄), 9.23 (s, pyr-H₂), 9.33 (d, pyr-H₆) ppm.

MS: m/z =635 [M+1 -Br]⁺.

Synthesis of Ibuprofen acid chloride: (±)Ibuprofen (2.95 grams, 14 mmol) or one of its optical isomers either R(−)-ibuprofen or S-(+)-ibuprofen, were placed in a dried, nitrogen-purged, 3-necked 100 ml flask. Oxalyl chloride (11 grams, 86 mmol) was then added dropwise, and the reaction was allowed to proceed at room temperature until evolution of gases ceased. Evaporation of the unreacted oxalyl chloride resulted in the formation of 3.2 grams of the product as a pale yellow solid (100% yield).

¹H-NMR (CDCl₃): δ=0.93 (d, (CH₃)₂, 6H), 1.61 (s, CH₃CH, 3H), 1.9 (m, CHCH₂, 1H), 2.5 (d, CHCH₂, 2H), 4.11 (q, CH₃CH, 1H), 7.19 (2d, Hα+Hβ, 4H) ppm.

Synthesis of Ibuprofen bromooctyl ester: Ibuprofen acid chloride (3 grams, 14 mmol) and 1-bromo-8-octanol (2.85 grams, 14 mmol), in acetonitrile (50 ml), were refluxed for 3 days. Evaporation of the solvent, followed by purification of the resulting crude oil by silica gel column chromatography, using a mixture of 1:1 ether/hexane, gave 4.4 grams of the product (83% yield).

¹H-NMR (CDCl₃): δ=0.89 (d, (CH₃)₂, 6H), 1.25 (m, CH₂, 10H), 1.48 (d, CH₃CH, 3H), 1.56 (quin, CH₂CH₂Br, 2H), 1.83 (m, CHCH₂, 1H), 2.45 (d, CHCH₂, 2H), 3.39 (t, CH₂Br, 2H), 3.68 (q, CH₃CH, 1H), 4.05 (t, OCH₂, 2H), 7.08 (d, Hα, 2H) 7.2 (d, Hβ, 2H) ppm.

Synthesis of N-(Ibuprofen octanoate)-3-N,N-dimethylcarbamoyl pyridinium bromide (IBU-PO): Ibuprofen bromooctyl ester (2.2 grams, 5.5 mmol) and 3-N,N-dimethylcarbamoyl pyridine (1.2 grams, 7.2 mmol), in methoxy ethanol (50 ml), were refluxed for 4 days. Evaporation of the solvent, followed by purification of the resulting crude oil by silica gel column chromatography, using gradient mixture of chloroform/methanol, gave 1.47 grams of the product (47% yield).

¹H-NMR (CDCl₃): δ=0.83 (d, (CH₃)₂, 6H), 1.25 (m, CH2, 12H), 1.42 (d, CH₃CH, 3H), 1.83 (m, CHCH₂, 1H), 2.38 (d, CHCH₂, 2H), 2.99 (s, (CH₃)₂N, 3H), 3.12 (s, (CH₃)₂N, 3H), 3.68 (t, CH₃CH, 1H), 3.97 (t, OCH₂, 2H), 4.91 (t, CH₂N, 2H), 7.03 (d, Hα, 2H), 7.14 (d, Hβ, 2H), 8.12 (t, pyr-H₅), 8.28 (d, pyr-H₄), 9.23 (s, pyr-H₂), 9.33 (d, pyr-H₆) ppm.

MS: m/z=483 [M-Br]⁺.

Synthesis of the optical isomer IBU-PO N-(S)-Ibuprofen (octanoate)-3-N,N-dimethylcarbamoyl pyridinium bromide (S-(+)-IBU-PO):

To a solid (S)-(+)-4-isopropyl-α-methyl-phenylacetic acid (0.540 gram, 2.62 mmol), oxalyl chloride (1.33 grams, 10.48 mmol) was gradually added, while stirring, at room temperature under nitrogen atmosphere. The obtained solution was stirred at room temperature until no evolution of gas was observed. The excess of oxalyl chloride was then removed at reduced pressure.

The obtained acid chloride was dissolved in dry dichloromethane (8.0 ml) and cooled (ice-water bath). A solution of 3-dimethylcarbamoyl-1-(8-hydroxy-octyl)-pyridinium bromide (0.82 gram, 2.19 mmol) and triethylamine (0.530 gram, 5.25 mmol) in dry dichloromethane (8.0 ml) was then added and the reaction mixture was stirred at room temperature overnight. Addition of ether, filtration of the obtained solid precipitate, washing the solid with ether and evaporation of the ether remnants gave a crude oil. Purification by silica gel column chromatography using a mixture of 5%/95% methanol/dichloromethane gave 0.573 gram of the product as an oil (46.6% yield).

¹H-NNR (CDCl₃): δ=0.89 (d(j=6.6 Hz), (CH₃)₂C), 1.11-1.44 (m, (CH₂)₄), 1.48 (d(j=7.2 Hz), CH₃CHCO₂), 1.55 (m, 2H), 1.83 (m, (CH₃)₂CH), 2.03 (m, 2H), 2.44 (d(j=7.1 Hz), (CH₃)₂CHCH₂), 3.06 (s, (CH₃)₂NCO), 3.18 (s, (CH₃)₂NCO), 3.68 (q(j=7.2 Hz), CH₃CHCO₂), 4.03 (t(j=6.7 Hz), CO₂CH₂), 5.00 (t(j=7.5 Hz), CH₂N⁺), 7.08 (d(j=8.0 Hz), 2 Ar-H), 7.19 (d(j=8.0 Hz), 2 Ar-H), 8.08 (dd(j=6.0,8.6 Hz), 1 pyr-H), 8.27 (d(j=8.6 Hz), 1 pyr-H), 9.27 (s, 1 pyr-H), 9.41 (d(j=6.0 Hz), 1 pyr-H) ppm.

Synthesis of Naproxen acid chloride: Naproxen (2.7 grams, 12 mmol) was placed in a dried, nitrogen-purged, 3-necked 100 ml round bottom flask. Oxalyl chloride (10.4 grams, 82 mmol) was then added dropwise, and the reaction was allowed to proceed at room temperature until evolution of gases ceased. Evaporation of the unreacted oxalyl chloride resulted in the formation of 2.9 grams of the product as a pale yellow solid (100% yield).

¹H-NMR (CDCl₃): δ=1.59 (d, CHCH₃, 3H), 3.91 (s, CH₃O, 3H), 4.15 (q, CHCH₃, 1H), 7.12 (m, nap-H₁+H₃, 2H), 7.4 (d, nap-H₄), 7.68 (m, nap-H₅+H₇+H₈, 3H) ppm.

Synthesis of Naproxen bromooctyl ester: Naproxen acid chloride (3 grams, 12 mmol) and 1-bromo-8-octanol (2.3 grams, 11 mmol), in acetonitrile (50 ml), were refluxed for 2 days. Evaporation of the solvent gave a white precipitate to which ether (50 ml) was added. The resulting ether solution was evaporated. Purification of the residual oil by silica gel column chromatography, using a mixture of 1:1 ether/hexane, gave 2.81 grams of the product (57% yield).

¹H-NMR (CDCl₃): δ=1.17 (m, CH₂, 8H), 1.29 (m, OCH₂CH₂, 2H), 1.59 (d, CHCH₃, 3H), 1.79 (m, CH₂CH₂Br, 2H), 3.35 (t, CH₂Br, 2H), 3.83 (q, CHCH₃, 1H), 3.89 (s, CH₃O, 3H), 4.05 (t, OCH₂, 2H), 7.1 (m, nap-H₁+H₃, 2H), 7.6 (m, nap-H₅+H₇+H₈, 3H) ppm.

Synthesis of N-(Naproxen octanoate)-3-(N,N-dimethylcarbamoyl) pyridinium bromide (NAPRO-PO): Naproxen bromooctyl ester (1.2 grams, 2.8 mmol) and 3-N,N-dimethylcarbamoyl pyridine (1 gram, 6 mmol), in methoxy ethanol (50 ml), were refluxed for 4 days. Evaporation of the solvent, followed by purification of the resulting crude oil by silica gel column chromatography, using a mixture of 17:3 chloroform/methanol, gave 0.87 gram of the product (52% yield).

¹H-NMR (CDCl₃): δ=1.22 (m, CH₂, 8H), 1.25 (m, OCH₂CH₂, 2H), 1.5 (d, CHCH₃, 3H), 1.9 (m, CH₂CH₂Br, 2H), 3.0 (s, CH₃N, 3H), 3.13 (s, CH₃N, 3H), 3.8 (q, CHCH₃, 1H), 3.87 (s, CH₃O, 3H), 4.01 (t, OCH₂, 2H), 4.9 (t, CH₂N, 2H), 7.1 (m, nap-H₇+H₃, 2H), 7.4 (d, nap-H₄), 7.6 (m, nap-H₅+H₇+H8, 3H), 8.16 (t, pyr-H₅), 8.25 (d, pyr-H₄), 9.15 (s, pyr-H₂), 9.24 (d, pyr-H₆) ppm.

Synthesis of Diclofenac bromooctyl ester: Diclofenac (0.97 gram, 3.27 mmol) and 1-bromo-8-octanol (2.9 grams, 13.8 mmol) were placed in a 50 ml flask. 37% Hydrochloric acid (2 ml) was added thereafter and the mixture was refluxed for 1 hour. The reaction was monitored by TLC and ¹H-NMR analysis. Chloroform (150 ml) was then added to the resulting yellow oil and the solution was dried (over anhydrous Na₂SO₄) and concentrated. Purification of the crude oil by silica gel column chromatography, using chloroform, gave 1.2 grams of the product (75% yield).

¹H-NMR (CDCl₃): δ=1.34 (m, CH₂, 8H), 1.65 (quin, CH₂CH₂Br, 2H), 1.84 (quin, OCH₂CH₂, 2H), 3.4 (t, CH₂Br, 2H), 3.81 (s, CH₂C(O), 2H), 4.14 (t, OCH₂, 2H), 6.57 (d, H₈), 6.98 (t, H₉+H₁₀, 2H), 7.13 (t, H₁₁), 7.25 (d, H₄), 7.34 (d, H₃+H₅, 2H) ppm.

MS (EI): m/z=487 [M⁺], 277, 242, 214.

Synthesis of N-(Diclofenac octanoate)-3-(N,N-dimethylcarbamoyl) pyridinium bromide (DICLO-PO): Diclofenac bromooctyl ester (0.460 gram, 0.94 mmol) and 3-dimethylcarbamoyl pyridine (0.5 gram, 3 mmol), in methoxy ethanol (10 ml), were refluxed for 4 days. Evaporation of the solvent, followed by purification of the crude oil by silica gel column chromatography using a mixture of 17:3 chloroform/methanol, gave 0.58 gram of the product, as a dark green oil (94% yield).

¹H-NMR (CDCl₃): δ1.28 (m, CH₂, 8H), 1.6 (quin, OCH₂CH₂, 2H), 2.01 (quin, CH₂CH₂N, 2H), 3.04 (s, CH₃N, 3H), 3.16 (s, CH₃N, 3H), 3.79 (s, CH₂C(O)O, 2H), 4.01 (t, OCH₂, 2H), 4.98 (t, CH₂N, 2H), 6.52 (d, H₈), 6.97 (t, H₉+H₁₀, 2H), 7.11 (t, H₁₁), 7.23 (d, H₄), 7.33 (d, H₃+H₅, 2H), 8.1 (t, pyr-H₅), 8.26 (d, pyr-H₄), 9.27 (s, pyr-H₂), 9.41 (d, pyr-H₆) ppm.

MS: m/z=572 [M-Br]⁺.

Synthesis of 3-(N,N-dimethylcarbamoyl)-1-(8-hydroxy-octyl) pyridinium bromide: 3-N,N-dimethylcarbamoyl pyridine (0.88 gram, 5.3 mmol) and 1-bromo 8-octanol (1.55 grams, 7.4 mmol), in methoxyethanol (80 ml), were refluxed for 2 days. Evaporation of the solvent followed by purification of the resulting crude oil by silica gel column chromatography, using a mixture of 1:4 methanol/chloroform, gave 0.62 gram of the product (31% yield).

¹H-NMR (CDCl₃): δ=1.29 (m, CH₂, 8H), 1.49 (m, HOCH₂CH₂, 2H), 2.05 (m, CH₂CH₂N, 2H), 3.03 (s, NCH₃, 3H), 3.16 (s, NCH₃, 3H), 3.57 (s, CH₂OH, 2H), 4.92 (t, CH₂N, 2H), 8.15 (t, pyr-H₅), 8.31 (d, pyr-H₄), 9.32 (s, pyr-H₂), 9.45 (d, pyr-H₆) ppm.

Synthesis of 3-N,N-Dimethylcarbamic acid 1-(8-hydroxy-octyl)-1,2,5,6-tetrahydro-pyridine-3-yl ester: Solid sodium borohydride (3.1 grams, 81.9 mmol) was added in portions during a period of 10 minutes into a cooled (ice-water bath) stirred solution of 3-N,N-dimethylcarbamoyl-1-(8-hydroxy-octyl)-pyridinium bromide (6.2 grams, 16.5 mmol) and methanol (150 ml). The cooled reaction mixture was stirred for 30 minutes followed by 1 hour stirring at room temperature. Evaporation of the solvent, dissolving the formed residue in water (35 ml), Extraction with 3×70 ml dichloromethane, drying over anhydrous MgSO₄ and concentration gave 4.5 grams of yellow oil. Purification by chromatography on a silica gel column, using a mixture of 95%/5% chloroform/methanol, gave 2.45 grams of the product, as a colorless oil (49.7% yield).

¹H-NMR (CDCl₃): δ=1.31 (s, (CH₂)₄, 8H), 1.54 (m, 4H), 2.25 (m, 2H), 2.44 (m, 2H), 2.59 (m, 2H), 2.93 (s, NCH₃, 3H), 2.95 (s, NCH₃, 3H), 3.00 (s, 2H), 3.62 (t, 2H), 5.44 (s, C═CH, 1H) ppm.

Synthesis of 2-(4-Isobutyl phenyl)-propionic acid 8-(5-dimethy lcarbamoyl-3,6-dihydro-2H-pyridine-1-yl)octyl ester (IBU-4H-PO): 2-(4-Isobutyl-phenyl)-propionyl chloride, prepared by reacting ibuprofen (0.560 gram, 2.72 mmol) with oxalyl chloride, and 3-N,N-dimethylcarbamic acid 1-(8-hydroxy-octyl)-1,2,5,6-tetrahydro-pyridine -3-yl ester (0.6 gram, 2.01 mmol), in dichloromethane (10 ml), were stirred at room temperature overnight. Dichloromethane (90 ml) was added thereafter to the reaction mixture. Washing with 6% aqueous sodium carbonate, drying the organic layer (over anhydrous MgSO₄) and evaporation of the solvent, gave crude yellow oil (0.98 grams). Purification by column chromatography on a silica gel (68 grams), using a mixture of 20%/80% methanol/chloroform, gave 0.382 gram of the pure product, as a colorless oil (39% yield).

¹H-NMR (CDCl₃): δ0.89 (d(j=6.6 Hz), (CH₃)₂CH, 6H), 1.24 (s, (CH₂)₄, 8H), 1.48 (d(j=7.2 Hz), CH₃CHCO₂, 3H), 1.55 (m, 4H), 1.83 (m, (CH₃)₂CH, 1H), 2.25 (m, 2H), 2.43 (2H), 2.44, (d, (CH₃)₂CHCH₂, 2H), 2.57 (t(j=5.7 Hz), CH₂N, 2H), 2.93 (s, N(CH₃), 3H), 2.94 (s, N(CH₃), 3H), 3.04 (s, 2H), 3.68 (q, CH₃CHCO₂, 1H), 4.04 (t(j=6.7 Hz), CO₂CH₂, 2H), 5.43 (bs, C═CH), 7.08 (d(j=8.0 Hz), Ar-2H), 7.20(d(j=8.0 Hz), Ar-2H) ppm.

MS: m/z=486 [M⁺], 414[M⁺-(CH₃)₂NCO, 100%].

Synthesis of 2-Acetoxy-benzoic acid 8-bromo-octyl ester (Aspirin bromooctyl ester): (a) 2-Acetoxybenzoyl chloride was synthesized as described by Liebeskind et al. (23). (b) 1-Bromo-8-octanol (3 grams, 14.3 mmol) and pyridine (2.94 grams, 37.2 mmol), in dichloromethane (45 ml), were stirred in a cooled ice-water bath, under N₂ atmosphere. A solution of 2-acetoxybenzoyl chloride (3.7 grams, 18.6 mmol) in dichloromethane (5 ml) was added gradually, and the reaction mixture was stirred at room temperature for 48 hours. After evaporation of the solvent, 300 ml ether were added, and washed with 2×50 ml water, 3×70 ml 0.3N dilute hydrochloric acid, 3×70 ml 5% aqueous sodium bicarbonate and 3×70 ml water. Drying the ether phase, concentration, extraction of the residue with 4×40 ml hexane and evaporation of the hexane extracts gave 4.7 grams of a viscous oil (88.3% yield).

¹H-NMR (CDCl₃): δ=1.30-1.50 (m, (CH₂)₄, 8H), 1.74 (m, 2H), 1.85 (m, 2H), 2.35 (s, OCOCH₃, 3H), 3.41 (t(j=6.8 Hz), CH₂Br, 2H), 4.27 (t(j=6.7 Hz), CO₂CH₂, 2H), 7.10 (dd, Ar-1H), 7.31 (dt, Ar-1H), 7.55 (m, Ar-1H), 8.02 (dd, Ar-1H) ppm.

Synthesis of 1-[8-(2-Acetoxy-benzoyl)-octyl]-3-N,N-dimethylcarbamoyl pyridinium bromide (ASP-PO): 2-Acetoxy-benzoic acid bromooctyl ester (1.01 grams, 2.73 mmol) and 3-N,N-dimethylcarbamoyl pyridine (0.56 gram, 3.37 mmol), in 2-methoxyethanol (25 ml), were refluxed overnight. Evaporation of the solvent, followed by ether extractions to remove residual solvent and non-polar impurities, gave 1.4 grams of insoluble oil. Purification by chromatography on a silica-gel column with a mixture of 95%/5% chloroform-methanol gave 0.84 gram of a viscous oil (57.5% yield).

¹H-NMR (CDCl₃): δ=1.24-1.50 (m, (CH₂)₄, 8H), 1.71 (m, 2H), 2.04 (m, 2H), 2.35 (s, OCOCH₃, 3H), 3.04 (s, CONCH₃, 3H), 3.17 (s, CONCH₃, 3H), 4.25 (t(j=6.7 Hz), CO₂CH₂, 2H), 4.99 (t(j=7.4 Hz), CH₂N⁺, 2H) 7.10 (dd, Ar-1H), 7.33 (dt, Ar-1H), 7.56 (m, Ar-1H), 8.01 (dd, Ar-1H), 8.10 (dd, Pyr-1H), 8.30 (m, Pyr-1H), 9.30 (s, Pyr-1H), 9.42 (d, Pyr-1H) ppm.

Synthesis of N-(8-hydroxyoctyl)-cytisine: 1-Bromo-8-octanol (0.198 gram, 0.947 mmol), cytisine (0.150 gram, 0.789 mmol), and potassium carbonate (0.120 gram, 0.868 mmol), in ethanol (4.0 ml) were refluxed overnight. Water (0.25 ml) and potassium carbonate (0.100 gram) were then added and the reaction mixture was shortly stirred. The solvents were thereafter removed under reduced pressure, and the residue was extracted with dichloromethane. Drying the organic extract over MgSO₄ and evaporation of the solvent gave a crude residue (0.290 grams)). Purification by silica gel column chromatography, using ethyl acetate, gave 0.189 gram of pure N-(8-hydroxyoctyl)-cytisine (75.3% yield).

¹H-NMR (CDCl₃): δ0.95-1.40 (m, 10H), 1.43-1.60 (m, 2H), 1.70-1.94 (m, 2H), 2.10-2.31 (m, 4H), 2.41 (brs, 1H), 2.80-3.00 (m, 4H), 3.63 (t(j=6.3 Hz), CH₂OH), 3.89 (m(j=1.1 Hz), 6.4 (d(j=15.4 Hz), 1H), 4.04 (d(j=15.4 Hz), 1H), 6.00 (dd(j=1.1,6.9 Hz), C═CH, 1H), 6.44 (dd(j=1.4,9.0 Hz), C═CH, 1H), 7.28 (dd(j=6.9,9.0 Hz), C═CH, 1H) ppm.

¹³C-NMR (CDCl₃): δ=25.6 (—CH₂—), 26.1 (—CH₂—), 26.4 (—CH₂—), 28.2 (—CH—), 29.1 (—CH₂—), 29.5 (—CH₂—), 33.1 (—CH₂—), 35.7 (—CH—), 50.3 (—CH₂—), 56.9 (—CH₂—), 60.3 (—CH₂—), 60.5 (—CH₂—), 62.7 (—CH₂—), 104.9 (—CH═), 116.4 (—CH═), 138.8 (—CH═), 152 (—C—), 163.8 (—C═O) ppm.

MS (EI): m/z=318 [M⁺, 288, 203 [M⁺-(CH₂)₇OH, 77%], 172, 160, 146, 117, 102, 84, 69, 58 (100%).

Synthesis of ibuprofen N-octyl-cytisine ester (IBU-OCT-CYTISINE): N-(8-hydroxyoctyl)-cytisine (0.150 gram, 0.472 mmol) and triethylamine (0.130 mg, 1.287 mmol), in dry dichloromethane (3.0 ml), were stirred in a cooled ice-water bath, under nitrogen atmosphere. A solution of 2-(4-isobutyl-phenyl)-propionyl chloride (the acid chloride of ibuprofen, 0.143 gram, 0.637 mmol) in dry dichloromethane (1 ml) was added gradually and the reaction mixture was stirred at room temperature overnight. The solvent was then removed under reduced pressure and water (5.0 ml) was added to the residue. A solution of concentrated potassium carbonate was added to adjust the pH to a value of 9-10, and the mixture was extracted with ether (3×20 ml). Drying the combined organic extracts over MgSO₄ and evaporation of the solvent gave 0.248 gram of crude oil. Purification by silica gel column chromatography, using a mixture of ethyl acetate/ether (1:5 v/v ratio) gave 0.187 gram of pure ibuprofen N-octyl-cytisine ester as a colorless oil (78.3% yield).

¹H-NMR (CDCl₃): δ=0.89 (d(j=6.6 Hz), (CH₃)₂C, 6H), 0.94-1.43 (m, 10H), 1.48 (d(j=7.1 Hz), CH₃CHCO₂, 3H), 1.40-1.64 (m, 2H), 1.70-1.95 (m, 2H), 2.10-2.32 (m, 4H), 2.40 (brs, 1H), 2.44 (d,(j=7.1 Hz), (CH₃)₂CHCH₂), 2.80-3.05 (m, 4H), 3.68 (q(j=7.1 Hz), CH₃CHCO₂, 1H), 3.87 (dd(j=6.6,15.4 Hz), 1H), 4.02 (d(j=15 Hz), 1H), 4.02 (t, CO₂CH₂, 2H), 5.96 (d(j=6.7 Hz), C═CH, 1H), 6.41 (d(j=9.0 Hz), C═CH, 1H), 7.08 (d(j=8.0 Hz), 2 Ar-H), 7.19 (d(j=8.0 Hz), 2 Ar-H), 7.25 (dd(j=6.8,9.0 Hz), C═CH, 1H) ppm.

¹³C-NMR (CDCl₃): δ=18.6 (CH₃CH), 22.5 (—(CH₃)₂CH—), 25.7 (—CH₂—), 26.1 (—CH₂—), 26.5 (—CH₂—), 26.8 (—CH₂—), 28.2 (—CH—), 28.5 (—CH₂—), 29.1 (—CH₂—), 29.2 (—CH₂—), 30.2 (—(CH₃)₂CH—), 35.7 (—CH-cytisine), 45.1 (—(CH₃)₂CHCH₂—), 45.3 (—CH₃CH—), 50.2 (—CH₂—), 57.5 (—CH₂—), 60.3 (—CH₂—), 60.5 (—CH₂—), 64.8 (—CH₂—), 104.5 (—CH═), 116.5 (═CH—), 127.2 (2 Ar—CH═), 129.3 (2 Ar—CH═), 138.0 (Ar—C—), 138.6 (—CH═), 140.4 (Ar—C—), 151.8 (—C—), 163.7 (—C═O), 174.9 (—CO₂—) ppm.

MS (EI): m/z=506 [M⁺], 491, 433, 398, 360, 318, 301, 204, 203 (100%), 161, 149.

Synthesis of 1-(8-Hydroxy-octyl)-3-methoxycarbonyl pyridinium bromide (or iodide): 1-Bromo-8-octanol (2.02 grams, 9.66 mmol), methyl nicotinate (5.13 grams, 37.45 mmol) and potassium iodide (1.62 grams, 9.76 mmol), in methanol (25 ml), were refluxed, under nitrogen atmosphere, overnight. Evaporation of the solvent, followed by two ether extractions to remove the residual solvent and starting materials, gave 4.2 grams of ether insoluble oil. Purification by silica gel column chromatography, using a mixture of 10%/90% methanol/chloroform, gave 2.7 grams of the product as a viscous yellow oil (71% yield).

¹H-NMR (CDCl₃): δ=1.2-1.6 (m, (CH₂)₅, 10H), 2.10 (m, 2H), 3.62 (t(j=6.4 Hz), CH₂OH), 4.08 (s, CO₂CH₃), 5.06 (t(j=7.6 Hz), CH₂N⁺), 8.38 (dd, Pyr-H), 8.97 (d(j=8.1 Hz), pyr-H), 9.43 (s, pyr-H), 9.94 (d(j=6.1 Hz), pyr-H) ppm.

Synthesis of 1-(8-Hydroxy-octyl)-1,2,5,6-tetrahydro-pyridine-3-carboxylic acid methyl ester (N-hydroxyoctyl-arecoline): Solid sodium borohydride (0.520 gram, 13.74 mmol) was added in portions into a cold (ice-water bath) stirred solution of 1-(8-hydroxy-octyl)-3-methoxycarbonyl pyridinium iodide (1.36 grams, 3.46 mmol) in methanol (40 ml), and the reaction mixture was stirred for 30 minutes. The solvent was then evaporated and the formed residue was dissolved in 30 ml water. Extraction with 200 ml ether, washing the organic layer with water (30 ml), potassium carbonate solution (2×30 ml) and water (2×30 ml), drying over MgSO₄ and concentration gave a crude yellow oil (1.10 grams). Purification by silica gel column chromatography using a mixture of 2% methanol in chloroform, gave 0.384 gram of the product (41.2% yield).

¹H-NMR (CDCl₃): δ=1.2-1.9 (m, (CH₂)₆), 2.38 (m, 2H), 2.47 (m, 2H), 2.55 (t(j=5.6 Hz), 2H), 3.20 (bs, NCH₂C═C), 3.66 (t(j=6.6 Hz), CH₂OH), 3.76 (s, CO₂CH₃), 7.02 (bs, CH═C) ppm.

Synthesis of 1-{8-[2-(4-Isobutyl-phenyl)-propionyl]-octyl}-1,2,5,6-tetrahydro-pyridine-3-carboxylic acid methyl ester (IBU-OCT-Arecoline, IOA): 2-(4-Isobutyl-phenyl)-propionyl chloride, formed by reacting ibuprofen (0.560 gram, 2.72 mmol) with oxalyl chloride, was added to 1-(8-Hydroxyoctyl)-1,2,5,6-tetrahydro-pyridine-3-carboxylic acid methyl ester (0.316 gram, 1.17 mmol) in dry dichloromethane (10 ml), and the reaction mixture was stirred at room temperature, under nitrogen atmosphere, overnight. Chloroform (70 ml) was added thereafter. Washing with 10% potassium carbonate solution (3×20 ml), drying the organic layer over MgSO₄ and evaporation gave a crude oil. Purification by a silica gel column chromatography, using a mixture of 0.8% methanol in chloroform, gave 0.37 gram of the pure product as a viscous oil (69% yield).

Optionally, the product can be obtained by sodium borohydride reduction of 1-{ 8-[2-(4-isobutyl-phenyl)-propionyl]-octyl}-3-methoxycarbonyl-pyridinium iodide.

¹H-NMR (CDCl₃): δ=0.89 (d(j=6.6 Hz), (CH₃)₂CH), 1.25 (m, (CH₂)₄), 1.48 (d(j=7.2 Hz), CH₃CHCO₂), 1.55 (m, 4H), 1.84 (m, (CH₃)₂CH), 2.35 (m, 2H), 2.44 (d(j=7.1 Hz), (CH₃)₂CHCH₂), 2.45 (m, 2H), 2.53 (t, 2H), 3.18 (bs, NCH₂—C═C), 3.68 (q(j=7.1 Hz), CH₃CHCO₂), 3.73 (s, CO₂CH₃), 4.04 (t(j=6.6 Hz), CO₂CH₂), 7.00 (bs, CH═C), 7.08 (d(j=8.1 Hz), Ar-2H), 7.20 (d(j=8.1 Hz), Ar-2H) ppm.

Synthesis of 1-{8-[2-(4-Isobutyl-phenyl)-propionyl]-octyl}-3-methoxycarbonyl-pyridinium iodide (IBU-OCT-methylnicotinate, IOMN): Ibuprofen-8-bromooctyl ester (0.547 grams, 1.38 mmol), methyl nicotinate (0.797 gram, 5.82 mmol) and potassium iodide (0.250 gram, 1.51 mmol), in methanol (10 ml), were refluxed, under nitrogen atmosphere, for 26 hours. Evaporation of the solvent and trituration of the residue with ether, gave an ether-insoluble residue which was dissolved in chloroform. Filtration to remove inorganic salts and evaporation gave 0.53 gram of crude yellow oil. Purification by chromatography on a silica gel column, using a mixture of 1:24 methanol/chloroform, gave 0.4 gram of the product (50% yield).

¹H-NMR (CDCl₃): δ=0.89 (d(j=6.6 Hz), (CH₃)₂CH), 1.17-1.45 (m, (CH₂)₅), 1.48 (d(j=7.1 Hz), CH₃CHCO₂), 1.55 (m, 2H), 1.84 (m, (CH₃)₂CH), 2.06 (m, 2H), 2.44 (d(j=7.2 Hz), (CH₃)₂CHCH₂), 3.68 (q(j=7.1 Hz), CH₃CHCO₂), 4.03 (t(j=6.7 Hz), CO₂CH₂), 4.08 (s, CO₂CH₃), 5.03 (t(j=7.6 Hz), CH₂N⁺), 7.08 (d(j=8.0 Hz), Ar-2H), 7.19 (d(j=8.0 Hz), Ar-2H), 8.37 (dd(j=6.1&8.0 Hz), pyr-H₅), 8.97 (d(j=8.0 Hz), pyr-H₄), 9.37 (bs, pyr-H₂), 9.93 (d(j=6. 1 Hz), pyr-H₆) ppm.

Synthesis of 7-Bromo-heptylamine: Bromo-heptanenitrile (1.50 gram, 7.89 mmol) in dry THF (40 ml) were cooled (ice-water bath) and stirred under nitrogen atmosphere. A borane solution in THF (1 M BH3-THF, 20 ml) was gradually added and the reaction mixture was stirred at room temperature overnight. After cooling the reaction mixture (ice-water bath), a solution of 1N HCl was added to achieve a pH value of 1-2 and cease the hydrogen evolution. A solution of 10% concentrated potassium carbonate solution was thereafter added to the reaction mixture (to achieve an alkaline Ph). Extraction with ether (3×35 ml), washing the combined ether extracts with concentrated potassium carbonate solution, drying over K₂CO₃ and evaporation of the solvent gave crude 7-bromo-heptylamine.

¹H-NMR (CDCl₃): δ=1.20-1.76 (m, 8H), 1.86 (m, CH₂CH₂Br), 2.72 (t(j=7.1 Hz), —CH₂NH₂), 3.41 (t(j=6.8 Hz), CH₂Br) ppm.

Synthesis of Ibuprofen 7-bromoheptyl amide: 7-Bromo-heptylamine was prepared as described hereinabove above and was immediately thereafter dissolved in dry dichloromethane (50 ml). Triethylamine (2.1 gram, 20.79 mmol) was added and the reaction mixture was cooled (water-ice bath) and stirred. 2-(4-Isobutyl-phenyl)-propionyl chloride (the acid chloride of ibuprofen) (2.5 gram, 11.13 mmol) was then added and the reaction mixture was stirred at room temperature for one day. Addition of ether (200 ml), washing with concentrated potassium carbonate solution, 1N HCl and water, drying the organic phase over MgSO₄ and evaporation of the solvent gave a crude oil. Purification by chromatography on a silica gel column, using mixtures of ether/hexane with increased ether concentrations followed by a mixture of 30%/70% ether/hexane gave 1.50 gram (49.7% yield) of pure ibuprofen-7-bromoheptyl amide as an oil, which crystallized upon standing (m.p. 39-40.5° C.

¹H-NMR (CDCl₃): δ=0.91 (d(j=6.6 Hz), (CH₃)₂CH—], 1.16-1.31 (m, 2(—CH₂—), 4H), 1.34-1.47 (m, 2(—CH₂—), 4H), 1.52 (d(j=7.2 Hz), CH₃CHCO₂, 3H), 1.82 (m, CH₂CH₂Br, 2H), 1.87 (m, (CH₃)₂CH, 1H), 2.47 (d(j=7.2 Hz), (CH₃)₂CHCH₂, 2H), 3.18 (m, CONHCH₂), 3.39 (t(j=6.8 Hz), CH₂Br, 2H), 3.53 (q(j=7.2 Hz), CH₃CHCON), 5.28 (brs, CONH), 7.13 (d(j=8.1 Hz), 2 Ar-H), 7.20 (d(j=8.1 Hz), 2 Ar-H) ppm.

MS(EI) m/z 381 and 383 (M⁺ very small); 302(M⁺-Br, 100%).

Synthesis of 3-Dimethylcarbamoyl-1-{7-[2-(4-isobutyl-phenyl)-propionylamino]-heptyl}-pyridinium bromide) (IBU-Heptylamide-PYR, IBU-Am-PH): A solution of ibuprofen-7-bromoheptyl amide (0.216 gram, 0.565 mmol) and 3-(N,N-dimethylcarbamoyl)pyridine (0.306 gram, 1.843 mmol), in 2-methoxy ethanol (5.0 ml), was refluxed overnight. Evaporation of the solvent and purification by silica gel column chromatography, using a mixture of 9%/91% methanol/chloroform gave 0.190 gram of the amide as an oil (61.4% yield).

¹H-NMR (CDCl₃): δ=0.89 (d(j=6.6 Hz), (CH₃)₂C, 6H), 1.15-1.53 (m, (CH₂)₄), 1.49 (d(j=7.2 Hz), CH₃CHCON), 1.83 (sep, (CH₃)₂CH), 2.04 (m, 2H), 2.43 (d(j=7.1 Hz), (CH₃)₂CHCH₂), 3.05 (s, (CH₃)₂NCO), 3.18 (s, (CH₃)₂NCO), 3.09-3.30 (m, CONCH₂), 3.63 (q(j=7.1 Hz), CH₃CHCO₂), 4.97 (t(j=7.6 Hz), CH₂N⁺), 6.00 (t, CONH), 7.08 (d(j=8.0 Hz), 2 Ar-H), 7.24 (d(j=8.0 Hz), 2 Ar-H), 8.06 (dd(j=6.0,8.7 Hz), 1 pyr-H), 8.28 (d(j=8.7 Hz), 1 pyr-H), 9.38 (s, 1 pyr-H), 9.49 (d(j=6.0 Hz), 1 pyr-H) ppm.

MS m/z 468.

The following equations (Scheme V) further illustrate the syntheses of the final products INDO-PO, IBU-PO, IBU-4H-PO, (S)-IBU-PO, NAPRO-PO, DICLO-PO, ASP-PO, IBU-OCT-Cytisine, IBU-OCT-Arecoline (IOA), IBU-OCT-methylnicotinate (IOMN), IBU-Heptylamide-Pyr (IBU-am-PH), and the various intermediates thereof:

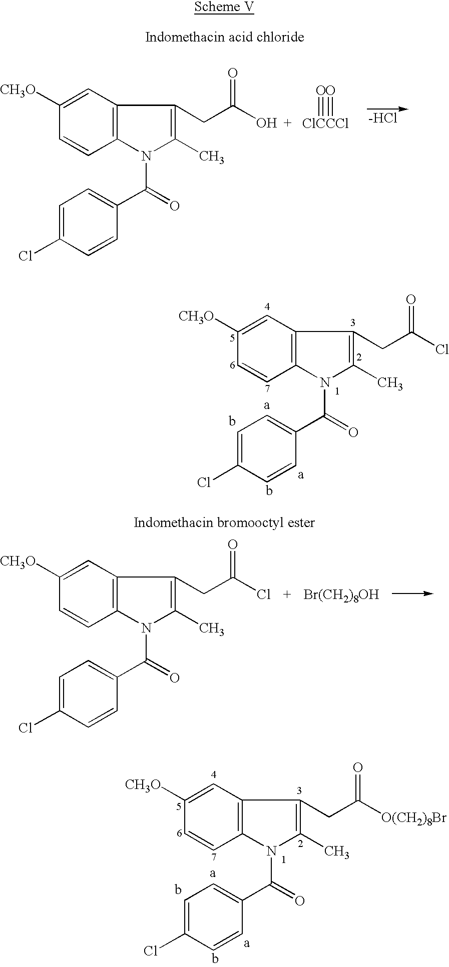

Activity Assays:

Inhibition of ChEs in vitro and in vivo: Purified recombinant human acetylcholineesterase (rHuAChE, 20 μl of 1.5 U/ml), fetal bovine serum-AChE (FBS-AChE 20 μl of 5 U/ml)) or purified human plasma butyrylcholinesterase (BChE) (HuBChE, 5 U/ml) was incubated in the presence of fixed concentrations (ranging from 0.01 μM to 0.6 μM) of NSAID-PYR-X compounds, IOA and IOMN at 25° C. Aliquots of such enzyme/inhibitor solution (20 μl) were transferred at specified time intervals into a cuvette containing 1.5 mM acetylthiocholine (ATCh, Sigma, USA) and 1.5 mM DTNB (Ellman reagent 29, Sigma, USA) in 1 ml phosphate buffer (50 mM, pH 7.4). The residual activity of AChE was measured by following the rate of increase in OD at 412 nm using a CARY 3 double-beam spectrophotometer (Varian, USA) (24).

In vivo inhibition of whole blood ChE in mice was measured by injecting the tested compounds to the animals and then sampling a 10 μl blood with a glass capillary from the eye orbit vein. The blood sample was transferred immediately thereafter into a cold water solution (0.09 ml, kept at 2-4° C.). The blood ChE activity was measured by transferring a 20 μl diluted blood sample into a 1 ml cuvette containing DTNB and ATCh in 1 ml phosphate buffer (50 mM, pH 7.4).

In vivo toxicity and Therapeutic Index: NSAID-PYR-X compounds were dissolved in 1:3 propylene glycol:water solution and were injected intramuscularly (0.1 ml per 25 grams) or intraperitoneally (0.2 ml per 25 grams) to mice. Toxic signs and mortality rates were observed during 24 hours. The LD₅₀ values were calculated according to Spearman Kerber method (25).

The Therapeutic Index (TI) for ChE inhibitors was calculated according to the following equation:

TI=LD₅₀/ED₅₀

Wherein:

LD₅₀ is the dose causing lethality to 50% of the animals: and

ED₅₀ is defined as the dose causing 50% ChE inhibition in blood.

Lipophilicity: A solution of 0.1 mM PYR-X, NSAID-PYR-X compound, IOA or IOMN in 1 ml n-octanol was prepared and placed in a plastic test tube. 1 ml phosphate buffer (50 mM, pH 7.4) was thereafter added and the mixture was stirred extensively by vortex for 2-3 minutes at 25° C. Phase separation at 2000 RPM for 10 minutes, was followed by transferring the upper n-octanol solution into a fused silica cuvettes and reading its OD at 272 or 333 nm. The selected wavelength was the λₘₐₓ of absorption according to the UV-visible spectra of a certain compound in n-octanol. The compound's concentration in n-octanol was determined by interpolation of the OD at λₘₐₓ, using a calibration curve for the specific compound in n-octanol. The partition coefficient (k) was calculated according to the following equation:

k=Cₒ/C_{b},

wherein,

Cₒ is the concentration of the compound in n-octanol

C_{b} is the concentration of the compound in phosphate buffer which was obtained by subtracting the observed Cₒ from the initial known concentration of the compound in n-octanol.

Inhibition of COX activity in vitro: The activity of cyclooxygenase I and II (COX I and COX II, Cayman Chemicals (MI, USA)) and the inhibition thereof by NSAIDs and NSAID-PYR-X compounds were measured by an immunoassay measuring prostaglandin E₂ (PGE₂) displacement. PGE₂ was produced in vitro from arachidonic acid (AA) by purified COX I or II. The assay was based on a competition binding technique in which PGE₂ competes with a fixed amount of alkaline phosphatase-labeled PGE₂ for sites of a mouse monoclonal antibody. During incubation, the mouse monoclonal antibody becomes bound to the goat anti-mouse antibody coated onto a microplate (R&D Systems, MN, USA). Following wash to remove excess conjugate and unbound sample, an alkaline phosphatase substrate solution was added to the wells to determine bound enzyme activity. Immediately following color development, absorbance at 405 nm was determined. The intensity of the color is inversely proportional to the concentration of PGE₂. The production of PGE₂ levels at the specified incubation time with COX I or COX II served as a quantitative assay for COX activity. Inhibition of COX activity was determined in the presence of various NSAID and NSAID-PYR-X compounds.

Peripheral anti-inflammatory activity: The anti-inflammatory activity of NSAID-PYR-X and NSAID compounds was evaluated by using the rat paw edema experimental model (26). Carrageenan (CAR, Sigma) solution (1% in saline) was intramuscularly injected into the rat hind paw, and the edema volume was measured quantitatively by using a plethysmometer (model 7140, Ugo Basile, Italy) attached to an electronic block reader (model 7141). A plethysmometer is a volume meter designed for accurate measurement of rat paw swelling. It consists of a water filled Perspex cell (2.5 cm in diameter) into which the rat paw is dipped up to a marked sign. The small difference in water level caused by volume displacement is recorded by a transducer coupled to a LCD read-out which shows the exact paw volume (control or treated). The maximal edema volume was obtained 2 hours after CAR injection. The efficacy of NSAID-PYR-X compounds against CAR-induced edema was measured by intraperitoneal injection of a NSAID-PYR-X compound 30 minutes prior to the CAR injection, followed by measurement of the edema volume 2 hours after the CAR injection. The level of edema (% EDEMA) was calculated according to the following equation:

% EDEMA=(Vₜ/V₀−1)−100,

wherein:

V₀=Pretreated rat paw volume (ml) (measurements which were taken before CAR injection); and

Vₜ=Treated rat paw volume (ml) (measurements which were taken 2 hours after CAR injection).

Anti-inflammatory activity in the brain:

Studies in mice: Male albino mice (25-30 grams) were injected with either 5 or 10 μl solution of either 1% CAR or saline solution, through the skull, into the left lateral ventricle, using a syringe equipped with a stainless steel needle that is 3 mm longer than a plastic spacer. The exact location for the injection into the left lateral ventricle was determined by measuring the specific coordinates using anatomical atlas of mouse brain and validated histologically by injection of 1% solution of Evans Blue dye in saline. Intraperitoneal NSAID-PYR-X treatment (5 or 10 mg/kg at 5 ml/kg) was applied 30 minutes before CAR injection. The mice were sacrificed 4 hours after the injection and their brains were isolated, dissected into four parts hemispheres and weighed immediately. The brain tissue was placed in a drying oven (140° C.) for 24 hours and weighed thereafter. The increase in brain water content due to CAR-induced inflammation, with and without treatment, was obtained by the following equation:

% Water Content=[(W₀−W₂₄)/W₀]×100,

wherein:

W₀=the weight before drying; and

W₂₄=the weight after drying for 24 hours.

These values served as a quantitative measure for brain edema induced by CAR injection. The decrease in the brain edema level after systemic intraperitoneal injection of an NSAID-PYR-X compound reflected its efficacy as an anti-inflammatory agent (27).

Studies in rats: Male Sprague-Dawley rats (300-420 grams) were anesthetized with equithisine solution and placed in a small animal stereotaxic instrument (Schuleler Co. Inc., NY, USA). The skull bone was exposed and a fixed cannule (Guide and Internal Cannule 6 mm, Bilaney, USA) was implanted into the left lateral ventricle. Solutions of either 1- % CAR or saline were injected through an internal cannule at a rate of 1 μl/minute using an electric pump (CMA 100 Microinjection Pump—Carnegie Medicin Stockholm, Sweden). NSAID-PYR-X treatment was applied intraperitoneally prior to the injection of CAR or saline, and the measurements were proceeded as described hereinabove.

Hypothermic effect: Male albino mice (25-35 grams) were placed in a constant temperature environment (22±1° C.). The mice were intraperitoneally injected either with 1:3 propylenglycol/water (PG:DDW) which served as a vehicle (carrier) or with a solution of IBU-PO dissolved in the 1:3 propylenglycol/water vehicle. The animals' rectal temperature was measured at specified time intervals after administration, using a bimetal rod thermometer coupled to Physitemp model BAT-12 reader (Physitemp Instruments, NJ, USA). In order to delineate the pharmacological mechanism of IBU-PO-induced hypothermia, either atropine (5mg/kg) or mecamylamine (2 mg/kg) were injected subcotenously 30 minutes before the IBU-PO injection, and the rectal temperature was monitored during the same specified time intervals.

Protection against closed head injury: Mice were subjected to a head injury which was caused by a stainless steel rod weighing 333 grams dropped from a height of 2 or 3 cm, and were intraperitoneally injected with various doses of PO, IBU-PO and NAPRO-PO (5, 7.5 and 10 mg/kg), 15 minutes thereafter. Clinical assessment of the animals was performed 1 hour post the head trauma using a neurological severity score (NSS) (28). Animals were examined by using specified motor tests wherein each failure in a particular test adds one point, while untreated animal has a score of 0. The NSS was determined again after 24 hours and the difference in NSS (ΔNSS) was calculated according to the following equation:

ΔNSS =NSS(t=1 h)−NSS(t=24 h)

Following the neurological score assessment, the mice were sacrificed and the affected hemispheres were weighed immediately thereafter and further after drying in oven for 24 hours at 140° C. The percentage of brain water content was calculated according to:

% Water Content=[(W₀−W₂₄)/W₀]×100

wherein,

W₀=the weight before drying; and

W₂₄=the weight after drying for 24 hours.

Protection against hypobaric hypoxia: Male albino mice (25-35 grams, n=4) were measured for their rectal temperature and were intraperitoneally injected thereafter either with 1:3 PG:DDW (vehicle) or with an IBU-PO dissolved in 1:3 PG:DDW. 30 minutes after the injection, rectal temperatures were measured for the indication of the drug effect, and the mice were placed in a glass dessicator at a pressure of 200 mm Hg for 15 minutes (29, 30) during which time of mice mortality was determined. The protection ratio (PR) of IBU-PO against hypobaric hypoxia was calculated using the following equation:

PR=t_{D}/t_{V}

wherein,

t_{D} is the time of death following drug treatment; and

t_{V} is time of death following injection with the vehicle.

Binding to rat brain muscarinic receptors: Rat brain homogenates were prepared according to a known procedure (31) and were stored at −70° C. until use. Competition binding of ligands to brain receptor was performed with specified concentrations of NSAID-PYR-X ligands in the presence of a fixed concentration of the following radioactive muscarinic antagonists: [³H]N-methylscopolamine (NMS) (for all muscarinic receptor subtypes), [³H]pirenzepine (selective for M1 receptors) and [³H]AFDX (selective for M2 receptors). The NSAID-PYR-X Ligands were incubated in the presence of the radioactive agonists and rat brain homogenate suspension (0.1 mg/ml protein in 50 mM Na/K phosphate buffer, pH 7.4) for 2 hours at 25° C.

Cholinergic muscarinic physiological response: Isolated guinea pig ileum was placed in a 5 ml glass bath filled with physiological Ringer solution (pH 7.4) kept at 37° C. by Haake thermostat. A constant stream of 95%/5% O₂/CO₂ was bubbled into the solution during the physiological measurement. Muscle contraction was induced by 0.5 μM carbamylcholine (CCh) and specified concentrations of NSAID-PYR-X were added thereafter. The antagonist effect of the tested compounds was determined by the decrease in CCh-induced muscle contraction which was caused by the addition of the tested compounds into the bath (32).

Stability of compounds in human plasma in vitro: NSAID-PYR-X compound IBU-PO (0.1 mM) was dissolved in 0.75 ml human plasma and incubated for specified time intervals at 37° C. The plasma solutions were separated thereafter by centrifugation (SA600 head, refrigerated Sorval centrifuge) at 15,000 rpm for 3 hours, using a Fugisep filtration membrane tubes (4 ml) with a cutoff number of 10 kDa. Water was then evaporated to dryness using a SpeedVac heated centrifuge under vacuum, and the residual oily material was treated with 1:1 acetonitrile:water solution and filtered through a membrane filter (Whatman, PURADISC 25AS 0.45μ filters). 50 μl of the filtered solutions were injected into HPLC column (RP-18 Merck, 125×4 mm) using isocratic carrier mixture of 75%/25% acetonitrile/phosphate buffer (5 mM, pH 7.4) containing 0.35 mM tetramethyl ammonium chloride. Quantitative analysis of the degradation product of IBU-PO, PO-OH, was performed using pyridostigmine as an internal standard. Electrospray Mass Spectrometric (ESMS) analysis was performed on parallel samples that were prepared similarly to the HPLC-injected samples.

### EXPERIMENTAL RESULTS

Inhibition kinetics of AChE and BChE in vitro: The kinetic parameters of the inhibition of purified rHuAChE, FBS-AChE and HuBChE by various NSAID-PYR-X compounds were measured and were further compared with the measured kinetic parameters of ChEs' inhibition by the known acetylcholine up regulators pyridostigmine (PYR) and Octyl-pyridostigmine (PO), and by the plasma hydrolysis product of IBU-PO (PO-OH) which was identified by ESMS analysis.

Table 1 below presents the inhibition kinetic parameters measured for IBU-PO, S-(+)-IBU-PO, IBU-am-PH, INDO-PO, NAPRO-PO, ASP-PO and DICLO-PO, as well as for PO-OH, PYR and PO. All NSAID-PYR-X compounds demonstrated inhibition of AChE and BChE with bimolecular rate constants (kᵢ) ranging from 5.4×10⁴ to 2.8×10⁶ M⁻¹ minute⁻¹. These rate constants are comparable with the values obtained with known ChEIs (e.g. PYR and PO). Moreover, the values obtained for the dissociation constants (K_{I}) indicate that the effective concentrations of most NSAID-PYR-X compounds range from 1.6×10⁻⁷ to 1.6×10⁻⁵ M and are thus similar to the values obtained for PYR. The value obtained for the optical isomer S-(+)-IBU-PO was, as predicted, significantly higher (2.2×10⁻⁴), while the value obtained for the chimeric compound that include an amide bond, IBU-am-PH, was somewhat lower. Nevertheless, these results suggest that the introduction of the NSAID moiety into the chimeric compound did not affect the potency of the pyridostigmine moiety toward the inhibition of AChE and/or BChE. Furthermore, the rate constants of the ChE's inhibition obtained by the plasma hydrolysis product PO-OH are comparable to those obtained by its parent chimer IBU-PO. These data demonstrate an inhibitory activity of both the prodrug (IBU-PO) and the released drug form (PO-OH).

TABLE 1

| | | K_{I} | K′ | kₛ × 10³ | t_{½}(kₛ) | kᵢ |
|---|---|---|---|---|---|---|
| INHIBITOR | ENZYME | (M) | (minutes⁻¹) | (minutes⁻¹) | (minutes) | (M⁻¹m⁻¹) |
| PYR | FBS-AChE | 1.2 × 10⁻⁶ | 0.31 | 10 | 69 | 2.5 × 10⁵ |
| | Hu-AChE | 1.5 × 10⁻⁶ | 0.40 | 9.2 | 75 | 2.7 × 10⁵ |
| | Hu-BuChE | 1.1 × 10⁻⁶ | 0.08 | 7.1 | 97 | 7.3 × 10⁴ |
| PO | FBS-AChE | 1.2 × 10⁻⁶ | 0.19 | 9.0 | 77 | 1.6 × 10⁵ |
| | Hu-AChE | 3.1 × 10⁻⁶ | 0.11 | 5.0 | 138 | 3.5 × 10⁴ |
| | Hu-BuChE | 2.0 × 10⁻⁶ | 0.09 | 6.2 | 111 | 4.0 × 10⁴ |
| PO-OH | FBS-AChE | 1.7 × 10⁻⁶ | 0.17 | 6.9 | 99.5 | 1.0 × 10⁵ |
| | Hu-AChE | 3.4 × 10⁻⁶ | 0.22 | 6.2 | 111 | 6.4 × 10⁴ |
| | Hu-BuChE | 9.2 × 10⁻⁷ | 0.05 | 0.6 | 1133 | 5.4 × 10⁴ |
| INDO-PO | FBS-AChE | 1.6 × 10⁻⁵ | 1.4 | 5.5 | 126 | 8.6 × 10⁴ |
| | Hu-AChE | 1.6 × 10⁻⁷ | 0.45 | 45 | 15.3 | 2.8 × 10⁶ |
| IBU-PO | FBS-AChE | 2.2 × 10⁻⁶ | 1.1 | 20 | 34 | 4.8 × 10⁵ |
| | Hu-AChE | 1.7 × 10⁻⁶ | 2.2 | 22 | 31 | 1.3 × 10⁶ |
| | Hu-BuChE | 7.5 × 10⁻⁶ | 1.5 | 3.6 | 192 | 2.0 × 10⁵ |
| S(+)IBU-PO | HUAChE | 1.2 × 10⁻⁶ | 0.32 | 29 | 24 | 2.7 × 10⁵ |
| IBU-4H-PO | FBS-AChE | 6.6 × 10⁻⁵ | 1.9 | 20 | 34.5 | 2.8 × 10⁴ |
| | Hu-AChE | 1.2 × 10⁻⁵ | 0.23 | 6.9 | 100 | 1.9 × 10⁴ |
| | Hu-BuChE | 1.6 × 10⁻⁶ | 0.19 | 0.9 | 789 | 1.2 × 10⁵ |
| IBU-Am-PH | HuAChE | 4.7 × 10⁻⁸ | 0.13 | 27 | 25.9 | 2.8 × 10⁶ |
| ASP-PO | FBS-AChE | 1.6 × 10⁻⁷ | 0.21 | 21 | 33 | 1.3 × 10⁶ |
| | Hu-AChE | 1.9 × 10⁻⁷ | 0.42 | 12.6 | 54.8 | 2.2 × 10⁶ |
| | Hu-BuChE | 4.9 × 10⁻⁷ | 0.04 | 0.7 | 1054 | 8.0 × 10⁴ |
| NAPRO-PO | FBS-AChE | 2.5 × 10⁻⁶ | 0.9 | 95 | 7.3 | 3.5 × 10⁵ |
| | Hu-AChE | 2.4 × 10⁻⁷ | 0.4 | 40 | 17.3 | 1.7 × 10⁶ |
| | Hu-BuChE | 2.1 × 10⁻⁷ | 0.07 | 1.1 | 627 | 3.3 × 10⁵ |
| DICLO-PO | FBS-AChE | 5.7 × 10⁻⁷ | 0.41 | 15 | 47 | 7.2 × 10⁵ |
| | Hu-AChE | 6.1 × 10⁻⁷ | 0.25 | 17 | 40 | 4.1 × 10⁵ |

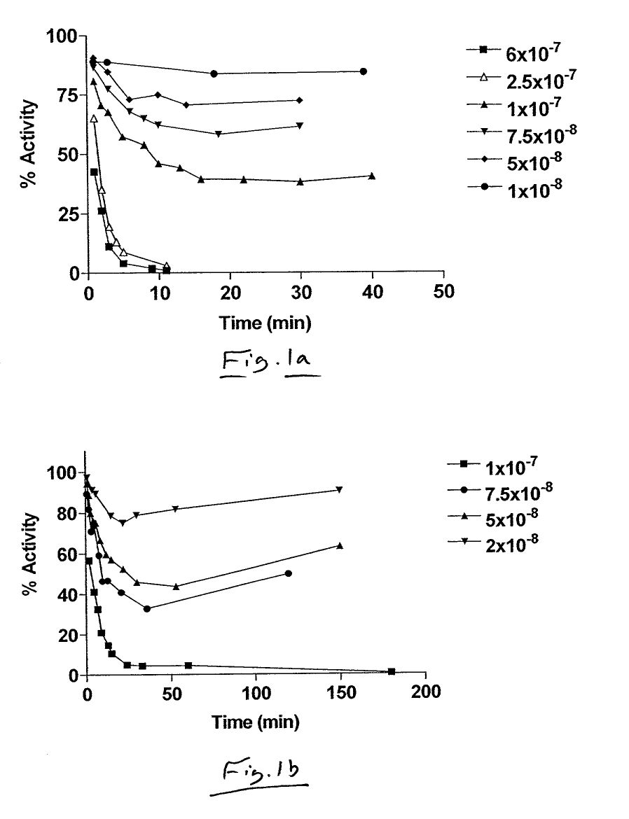

FIG. 1 demonstrates the time-course of inhibition of FBS-AChE and HuAChE by various concentrations of DICLO-PO. Inhibition kinetics is similar to that obtained with other known carbamate derivatives, approaching a steady-state at time periods that depend on the carbamate derivative concentration.

The kinetic measurements performed with arecoline, methyl nicotinate (MN), and the chimeric compounds IOA and IOMN, surprisingly showed that these compounds demonstrate reversible inhibition of AChE.

Table 2 below presents the IC₅₀ values obtained for AChE inhibition in the presence of these compounds. The data show that the chimeric compound IOMN demonstrates reversible inhibition of AChE with an IC₅₀ value that is comparable to the K_{I} value obtained with pyridostigmine (see Table 1 hereinabove), whereas both methyl nicotinate (MN) and Arecoline demonstrate much lower activity as AChE inhibitors. However, the IC₅₀ value obtained with the chimeric compound IOA shows that the reversible AChE inhibition activity thereof is one order of magnitude lower than that obtained with IOMN.

TABLE 2

| Inhibitor | Enzyme | IC₅₀ (M) |
|---|---|---|
| MN | Hu-AChE | >10⁻⁴ |
| IOMN | Hu-AChE | 3.3 × 10⁻⁶ |
| Arecoline | Hu-AChE | >10⁻⁴ |
| IOA | Hu-AChE | 7.3 × 10⁻⁵ |

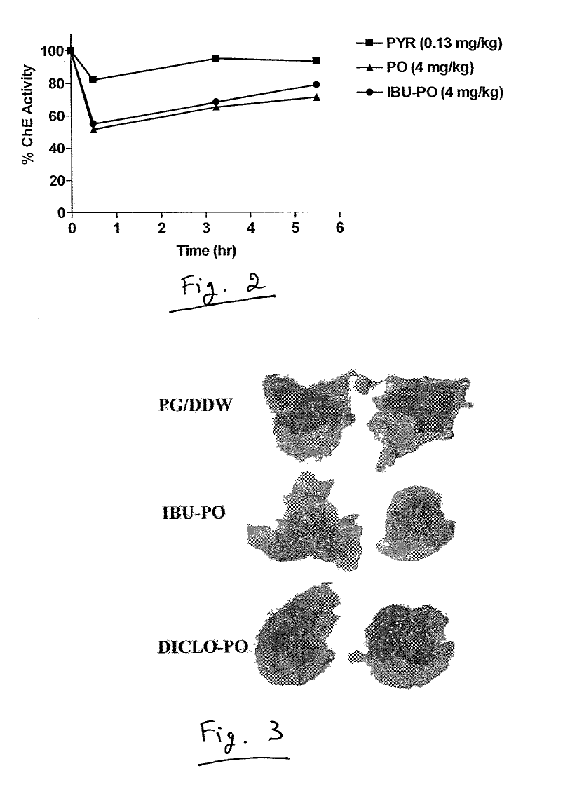

Inhibition kinetics of AChE and BChE in vivo: FIG. 2 shows the time-course of whole blood ChE inhibition following the injection of PYR, PO or IBU-PO in mice. Using doses of 4 or 10 mg/kg of PO or IBU-PO resulted in 30-50% ChE inhibition, for a period of at least 5 hours. In particular, intraperitoneal injection of 4 mg/kg IBU-PO resulted in 50% inhibition of blood ChE within 30 minutes, with a duration time of several hours. In contrast, using 0.13 mg/kg PYR resulted in less than 20% ChE inhibition, with a much shorter duration time (1-1.5 hour) than that observed for IBU-PO. These data indicate that the chimeric compound IBU-PO demonstrates both longer duration of action in vivo and higher Therapeutic Index (as is described herein below), as is compared with PYR.

In vivo toxicity and therapeutic index: Table 3 below presents the LD₅₀ values obtained for PYR, PO, NSAIDs, NSAID-PYR-X compounds and IOMN in mice. All the NSAID-PYR-X compounds are 10-50 fold less toxic than PYR, having LD₅₀ values that range from 22.5 to 57.4 mg/kg of intramuscular injection. The least toxic NSAID-PYR-X compounds are IBU-PO and DICLO-PO, having LD₅₀ values of 57.4 and 53.4 mg/kg (intramuscular) and 61.2 and 53.4 mg/kg (intraperitoneal), respectively. Thus, further studies were pursued mainly with IBU-PO, due to its lower toxicity and outstanding activity in some of the pharmacological models described below. However, the chimeric compound of IBU and methyl nicotinate, IOMN, was found to be even less toxic than these NSAID-PYR-X compounds, having a LD₅₀ value greater than 100 mg/Kg (intraperitoneal).

TABLE 3

| | LD₅₀ | LD₅₀ |
|---|---|---|
| | (mice i.m.) | (mice i.p.) |
| COMPOUND | mg/kg | mg/kg |
| PYRIDO | 2.1 | 5.1 |
| | (1.9-2.3) | (4.7-7.8) |
| PO | 37.6 | 49.5 |
| | (26.6-52.6) | (30.6-80.0) |
| PO-OH | 4.32 | |
| | (3.1-6.1) | |
| INDO-PO | | 35 |
| | | (29.1-42.6) |
| INDO | — | 12.4 |
| | | (9.4-16.7) |
| IBU | >100 | — |
| IBU-PO | 57.4 | 61.2 |
| | (43.9-75.0) | (50.8-73.6) |
| IOMN | | >100 |
| IBU-4H-PO | | 54.22 |
| | | (13.9-211.4) |
| DICLO-PO | 53.4 | 53.4 |
| | (38.0-75.0) | (38.8-75.0) |
| NAPRO-PO | | 37.2 |
| | | (25.2-55.1) |
| ASPIRIN | | >100 |
| ASP-PO | | 47.7 |
| | | (39.1-58.1) |
| PD | 36.59 | |
| | (25.5-52.5) | |
| IBU-PD | 22.5 | |
| | (18.4-27.4) | |

Furthermore, as is shown in FIG. 3, intraperitoneal injection of 10 mg/kg of IBU-PO and DICLO-PO did not cause any detrimental effect on the mucosal tissue of rat stomach. It is well established in the prior art that NSAID such as ibuprofen and indomethacin cause erosions and bleeding ulcers in the gastrointestinal system. Thus, these observations are consistent with the notion that the esterification of the carboxylic acid group in NSAID's markedly reduces the gastrointestinal side effects thereof (33).

The Therapeutic Index (TI) is defined by the following equation:

$$TI = \frac{{LD}_{50} ( i . p . mice )}{{ED}_{50} ( ChE Inhibition in mouse blood )}$$

The TI calculated for IBU-PO in mice is 15.3 (=61.2/4). This value is significantly higher than TIs calculated for the known ChE inhibitors physostigmine, TACRINE and EXELON, which are 3.4, 3.5 and 12.5, respectively.

Lipophilicity: The lipophilicity of the compounds of the present invention was measured by phase partition experiments (n-octanol and phosphate buffer). Table 4 below presents the partition coefficient (kₚ) values between n-octanol and phosphate buffer of PO, PO-OH, NSAID-PYR-X compounds, IOMN and IOA. The kₚ values obtained for IBU-PO, IBU-4H-PO, NAPRO-PO and INDO-PO are significantly higher than those obtained for PO and PO-OH. Furthermore, the Kₚ value obtained for the tertiary amine form IBU-4H-PO is much higher than its quaternary congener IBU-PO. However, it was unexpectedly found that the Kₚ value obtained for the quaternary ammonium IOMN (Kₚ=28.4, see Table 4) is much higher than the value obtained for its tertiary amine derivative IOA (Kₚ=4).

TABLE 4

| Compound | Kₚ |
|---|---|
| PO | 0.53 |
| PO-OH | 0.53 |
| INDO-PO | >99 |
| IBU-PO | 6.14 |
| IBU-4H-PO | 99 |
| ASP-PO | 0.47 |
| NAPRO-PO | 6.5 |
| IOMN | 28.4 |
| IOA | 4 |

Inhibition of cyclooxygenase (COX) isoenzymes in vitro: The inhibition of constitutive cyclooxygenase (COX I) and its inflammation inducible isoenzyme COX II by NSAIDs and NSAID-PYR-X chimeric compounds was measured using a quantitative competitive PGE₂ immunoassay. Purified COX I (bovine seminal vesicles) and COX II (sheep placenta) were used for the assay. Table 5 below presents the inhibition level of COX I and COX II obtained with 1 μM of NSAIDs and NSAID-PYR-X compounds. The data are consistent with the prior art as to the selectivity of ibuprofen, diclofenac and, to some extent, indomethacin toward COX II. The chimeric compounds IBU-PO and INDO-PO further demonstrate some selectivity toward COX II as compared to COX I. The data further show that NSAID-PYR-X chimera are effective COX I and COX II inhibitors at concentrations that are equivalent to those of NSAIDs (about 1 μM). Moreover, these concentrations are similar to the K_{I} values of AChE and BChE inhibition accepted for the chimeric compounds as is presented in Table 1 hereinabove.

TABLE 5

| | |
|---|---|
| Compound | % Inhibition |

| 10⁻⁶ M | COX I | COX II |
|---|---|---|
| IBU-PO | 41 | 62 |
| INDO-PO | 41 | 75 |
| NAPRO-PO | 55 | 62 |
| IBU | 35 | 68 |
| INDO | 29 | 73 |
| NAPRO | 37 | 85 |
| DICLO | 39 | 85 |

Peripheral anti-inflammatory activity: The effect of NSAIDs and NSAID-PYR-X compounds on CAR-induced rat paw edema was evaluated by intraperitoneally injecting the compounds 30 minutes before CAR and measuring the change in paw volume 2 hours after CAR injection.

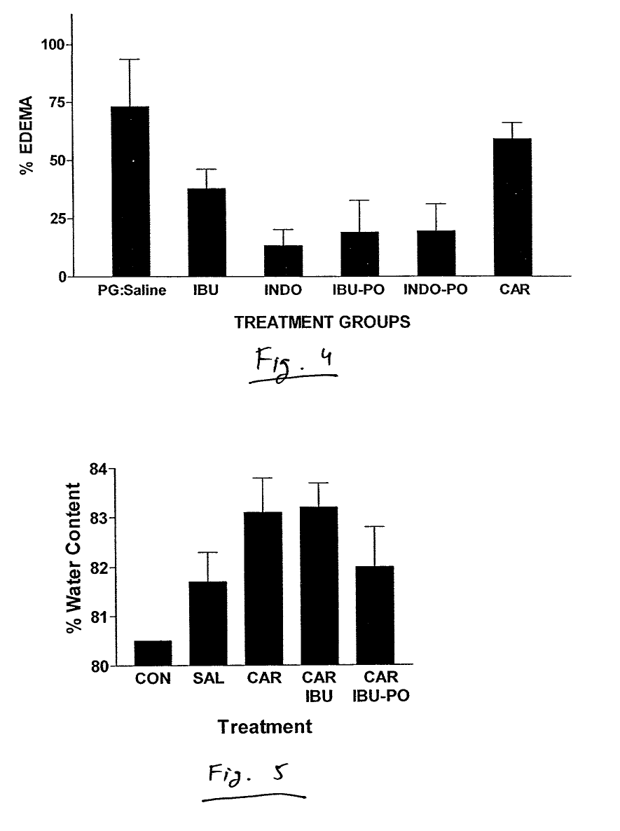

FIG. 4 shows the effect of the NSAIDs IBU and INDO, and the chimeric compounds IBU-PO and INDO-PO on carrageenan (CAR)-induced rat paw edema, as well as the effect of injecting the vehicle (1:3 PG/DDW) only. The results demonstrate that injecting 5 mg/kg of all compounds resulted in the same significant decrease in edema level (to about 30%) as compared to injecting the vehicle only, thus indicating that the new NSAID-PYR-X chimeric compounds display peripheral anti-inflammatory activity that is comparable to that of clinically used NSAIDs. Furthermore, it should be noted that the chimeric compounds were injected together with a carrier which by itself induce larger edema then CAR without any treatment (73 vs. 59%, respectively). It is therefore concluded that these chimeric compounds display higher anti-inflammatory activity than their corresponding NSAID compounds.

Anti-inflammatory activity in brain: The effect of IBU and IBU-PO on CAR-induced brain edema in rats, was measured following the injections thereof 10 minutes prior to intra cerebral ventricle (icv) (left lateral ventricle) injection of CAR (1% solution in saline).

FIG. 5 demonstrates the effect of IBU and IBU-PO on the water content in rats' brain. While pretreatment with IBU resulted in no change in the edema water content elicited by CAR (83%), the pretreatment with the chimeric compound IBU-PO reduces the % water content to 82% which is similar to that obtained after saline icv injection, indicating its capability to cross the BBB and act as an anti-inflammatory drug.

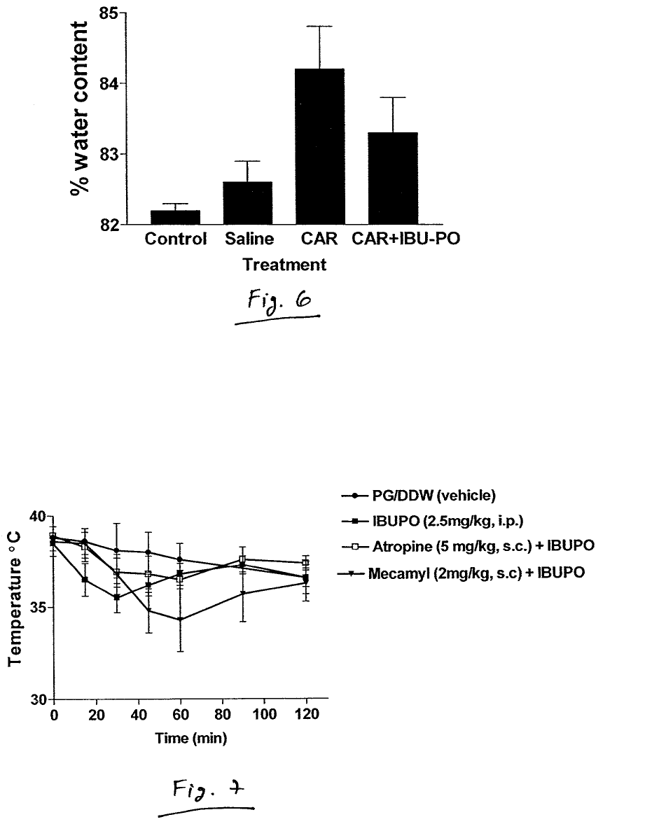

FIG. 6 demonstrates the effect of IBU-PO on CAR-induced brain edema in mice. Intraperitoneal injection of 10 mg/kg IBU-PO significantly reduces the brain water content in mice, thus indicating its efficacy as a central anti-inflammatory drug in both mice and rats.

Hypothermic effect: FIG. 7 shows the time-course of hypothermia induced by IBU-PO in mice. Intraperitoneal injection of 2.5 mg/kg IBU-PO resulted in an IBU-PO-induced hypothermic effect which is maximal at 30 minutes and persists up to at least 6 hours following injection. Pretreatment with 5 mg/kg atropine applied subcotenously partially reverses the hypothermia, whereas pretreatment with 2 mg/kg of the nicotinic antagonist mecamylamine applied subcotenously cause a significant delay in the maximal hypothermia induced by IBU-PO from 30 to 60 minutes.

Protection against closed head injury: The neuroprotective activity of the novel chimeric compounds was evaluated using a closed head injury model in mice (28), which were injected with various doses of PO, IBU-PO and NAPRO-PO 15 minutes after subjection to a head injury.

Table 6 below presents the percent water content and difference in neurological severity score (ΔNSS) of the animals with and without treatment, compared with sham animals with no head injury. As a rule, larger ΔNSS value represents higher drug efficacy against the damage induced by the closed head injury.

FIG. 8 demonstrates the effect of NSAID-PYR-X compounds on the edema level caused by a head injury.

The values presented in Table 6 and FIG. 8 show the high efficacy demonstrated by IBU-PO (water (%): 80.94, 81.45 and 80.91 and ΔNSS: 1.25, 2.0 and 2.5) as compared to the known ChEI derivative PO (water (%): 81.17, 82.77 and 83.43 and ΔNSS: 1.25, 1.5 and 1.4) at doses of 5, 7.5 and 10 mg/kg, respectively. The water content and ΔNSS values obtained for IBU-PO are comparable to those obtained for the most efficacious neuroprotective compounds tested so far in this model (e.g., TEMPOL and HU-21 1). NAPRO-PO demonstrates a moderate protective activity against head trauma (water (%): 81.38, 82.09 and 81.38 and ΔNSS: 2.0, 1.52 and 2.25) at the same respective doses as mentioned above. Nevertheless, its efficacy is still higher then PO.

TABLE 6

| | | Water | |
|---|---|---|---|
| | Dose | content | |
| Treatment | (mg/kg) | (%) | DNSS |
| Sham | | 78.65 ± 0.62 | 0 |
| Vehicle only | | 83.64 ± 0.07 | 1.0 ± 0.21 |
| PO | 5 | 81.17 ± 0.72 | 1.25 ± 0.63 |
| | 7.5 | 82.77 ± 0.66 | 1.50 ± 0.76 |
| | 10 | 83.43 ± 0.64 | 1.40 ± 0.51 |
| IBU-PO | 5 | 80.94 ± 0.84* | 1.25 ± 0.63 |
| | 7.5 | 81.45 ± 0.35 | 2.00 ± 0.8^{#} |
| | 10 | 80.91 ± 0.47* | 2.50 ± 0.85* |
| NAPRO-PO | 5 | 81.38 ± 0.59 | 2.00 ± 1.0 |
| | 7.5 | 82.09 ± 0.19 | 1.50 ± 0.62 |
| | 10 | 81.38 ± 0.55 | 2.30 ± 0.48* |

*p < 0.05 vs vehicle treated (PG:DDW, 1:3);
^{#}p = 0.058 vs vehicle treated

Protection against hypobaric hypoxia: The neuroprotective, anti-ischemic and anxiolytic activity of the chimeric compounds of the invention was evaluated using the hypobaric hypoxia model in mice (29, 30), by measuring their effect on survival time of mice during hypobaric hypoxia.

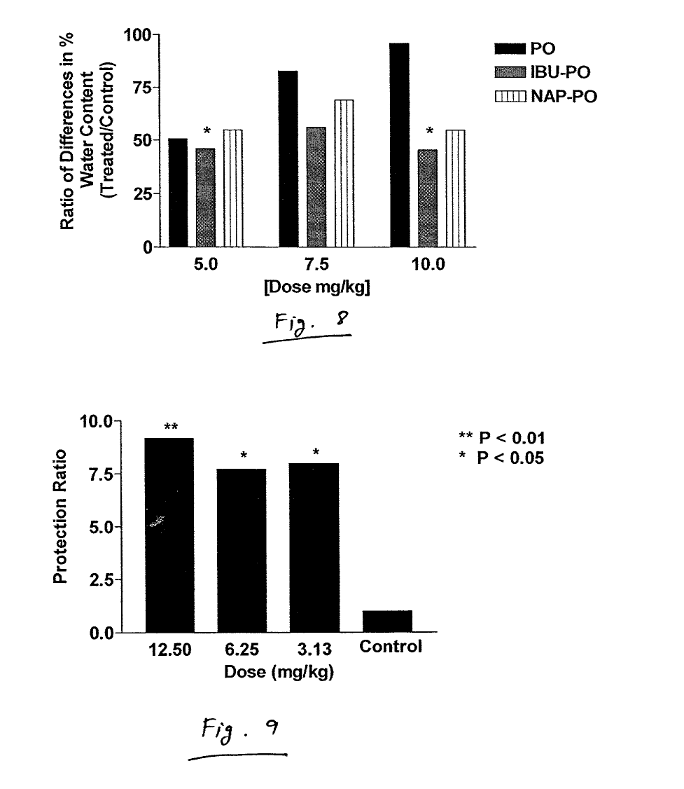

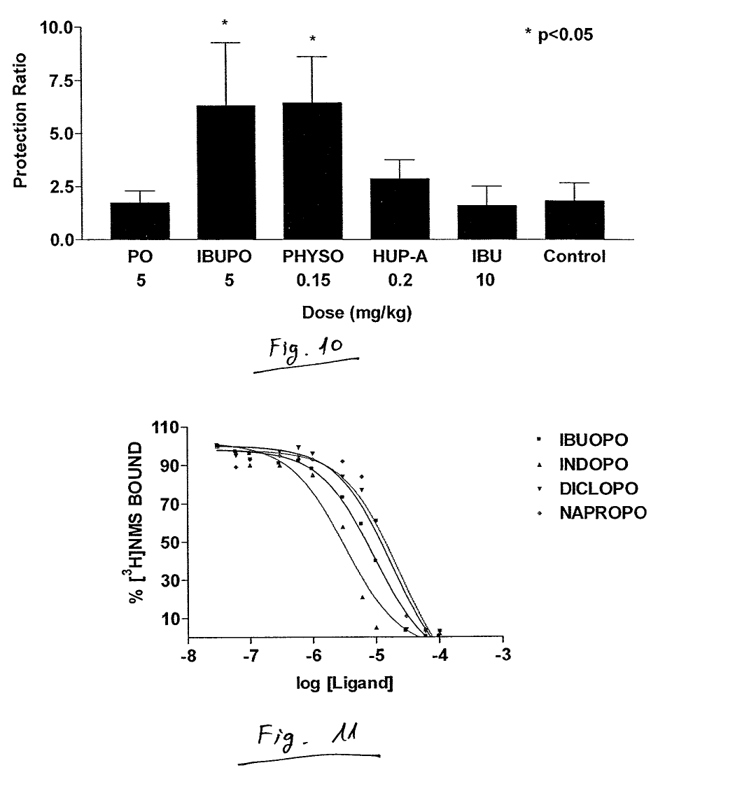

FIG. 9 demonstrates the effect of injecting various doses of IBU-PO to mice, prior to induction of hypobaric hypoxia, compared with a control group injected with a vehicle only. The protection of IBU-PO against hypoxia is 7.5-9 fold larger than that of the vehicle control (100 seconds). Moreover, some of the mice treated with IBU-PO survived even the maximal hypoxia period of 15 minutes.

FIG. 10 shows, in comparison, the effect of other known ChE inhibitors, e.g., physostigmine (0.15 mg/kg injected intramuscularly), huperzine-A (0.2 mg/kg injected intramuscularly) PO (5 mg/kg injected intraperitoneally) and ibuprofen (0.10 mg/kg injected intraperitoneally) on hypoxia in mice. The only ChEI that provides good protection against hypoxia is physostigmine, suggesting that ChE inhibition by itself does not necessarily provide protection against hypoxia. Hypothermic effect of 2-3° C. was observed after injection of IBU-PO and physostigmine. However, it was previously observed that the anti-hypoxia effect induced by ChE inhibitors and cholinergic agonists is not attributed solely to hypothermia but rather to direct neuronal effect (34).

Binding to brain cholinergic receptors: The novel NSAID-PYR-X chimeric compounds were tested for their interaction with cholinergic muscarinic receptor subtypes by measuring their competitive displacement of radioactive muscarinic ligands from rat brain in vitro.

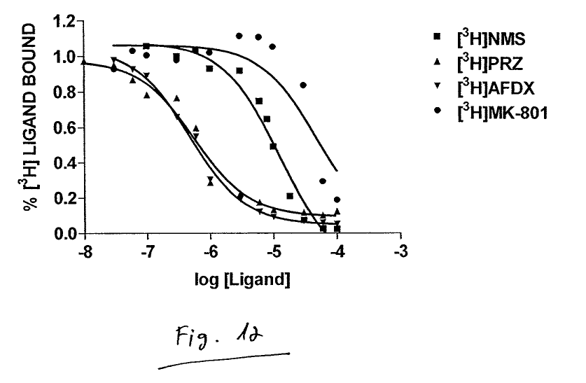

FIG. 11 demonstrates the binding curves for the displacement of [³H]NMS from rat brain by NSAID-PYR-X compounds.

Table 7 below presents the IC₅₀ and K_{I} values obtained for IBU-PO, INDO-PO, DICLO-PO and NAPRO-PO. The K_{I} values range from 1.0×10⁻⁶ to 6.9×10⁻⁶ M, whereas the effective concentrations obtained for the interaction of the NSAID-PYR-X chimeric compounds with the muscarinic receptors are similar to those received for ChE and COX inhibition, as described hereinabove.

Table 8 below presents the binding parameters of IBU-PO to M1 and M2 muscarinic receptors, using [³H]pirenzepine and [³H]AFDX-346 as the specific labeled ligands, as well as [³H]MK-801 for NMDA receptors. The K_{I} values obtained for IBU-PO from the displacement of [³H]pirenzepine and [³H]AFDX-346 are 5.1×10⁻⁷ and 4.4×10⁻⁷ M, respectively, indicating that IBU-PO binds at the same affinity to both M1 and M2 muscarinic receptor subtypes. IBU-PO demonstrates lower affinity toward NMDA receptors as evidenced from the competition with tritiated MK-801 (K_{I}=4.3×10⁻⁵ M).

FIG. 12 shows all respective binding curves for IBU-PO with the various radioactive receptor ligands in rat brain.

TABLE 7

| COMPOUND | IC₅₀ (M) | K_{I} (M) |
|---|---|---|
| IBU-PO | 9.6 × 10⁻⁶ | 2.8 × 10⁻⁶ |
| INDO-PO | 3.3 × 10⁻⁵ | 9.6 × 10⁻⁷ |
| DICLO-PO | 1.8 × 10⁻⁵ | 5.3 × 10⁻⁶ |
| NAPRO-PO | 2.3 × 10⁻⁵ | 6.9 × 10⁻⁶ |

K_{D} [³H]NMS=0.2 nM, dissociation constant of radioactive ligand.

L [³H]NMS=0.48 nM, concentration of radioactive ligand.

TABLE 8

| | K_{D} | L | IC₅₀ | K_{I} |
|---|---|---|---|---|
| [³H]LIGAND | (nM) | (nM) | (M) | (M) |
| NMS | 0.2 | 0.65 | 1.3 × 10⁻⁵ | 2.9 × 10⁻⁶ |
| AFDX-346 | 5 | 0.49 | 4.8 × 10⁻⁷ | 4.4 × 10⁻⁷ |
| PIRENZEPINE | 13.9 | 1.7 | 5.7 × 10⁻⁷ | 5.1 × 10⁻⁷ |
| MK-801 | 33 | 4.8 | 4.9 × 10⁻⁵ | 4.3 × 10⁻⁵ |

Stability in human plasma in vitro: The rate of hydrolysis of NSAID-PYR-X chimeric compounds in human plasma was evaluated by incubating IBU-PO in human plasma at 37° C., in vitro, for various time intervals, in the presence of pyridostigmine as an internal standard.

The estimated half-life time (t_{½}) for the hydrolysis of IBU-PO in plasma at 37° C., based on HPLC analysis, is 4.5-5 hours.

The degradation products of the hydrolysis of IBU-PO, at various time intervals in plasma, are PO-OH and ibuprofen, as was determined by quantitative electrospray mass spectrometry (ESMS) analysis, using pyridostigmine as an internal standard.

The ESMS spectra obtained for the degradation products show unequivocally that the dimethylcarbamoyl moiety in the ChEI derivative remains attached to the pyridine ring during hydrolysis of the chimeric compound in plasma.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### References

1. Giacobini, E., Jpn. J. Pharmacol., 74, 225-241 (1997).

2. Fisher, A., Brandeis, R., Haring, R., Eshhar, N., Heldman, E., Karton, Y., Eisenberg, O., Meshulam H., Marciano, D., Bar-Ner N. and Pittel, Z., J. Physiol Paris, 2(5-6):337-40 (1998).

3. Thal, I. J., Forrest, M., Loft H. and Mengel, H., Neurology, 54, 421-426 (2000).

4. Sabb, A. L., Husbands, G. M., Tokolics, J., Stein, R. P., Tasse, R. P., Boast C. A., Mayer, J. A. and Abou Gharbia, M., Biorg. Med. Chem. Lett., 9(4), 1895-1900 (1999).

5. Scwartz, R. D., Callahan, M. J., Coughenour, L. L., Dickerson, M. R., Kinsorra, J. J., Lipinski, W. J., Raby C. A., Spencer, C. J. and Tecle, H., J. Pharmacol. Exp. Ther., 291, 812-822 (1999).

6. Giacobini, E., in: Alzheimer Disease: From Molecular Biology to Therapy, Edited by Becker, R. and Giacobini, E., pp. 187-204, Birkhauser, Boston (1996).

7. Clementi, F., Fomasari, D. and Gotti, C., Trends Pharmacol. Sci., 21, 35-37 (2000).

8. Amitai, G., Adani, R., Rabinovitz, I., Sod-Moriah, G., Rachaman, E., Brandeis, R. and Heldman, E., 5th International meeting on Cholinesterases, San Diego, Calif., Structure and Function of Cholinesterases and Related Proteins Eds. Doctor, B. P., Taylor, P., Quinn, D. M., Rotundo, R. L. and Gentry, M. K. Plenum Press (New York and London) PP. 277-282 (1998)

9. Inestrosa, N. C. Alvarez, A. and Kalderon, F. Molec. Psychiat. 1, 359-361 (1996) and Alvarez, A., Opazo, C., Alarcon, R., Garrido, J. and Inestrosa, N. C. J. Mol. Biol., 272, 348-361 (1997).

10. Holloway, H. W. Utsuki, T. Brossi, A. and Greig, N. H., J. Med. Chem., 42, 1855-65 (1999)

11. Breitner, Neurobiol. Aging, 17(5), 789-794, (1996).

12. Hull M., Fiebich B. L., Lieb K., Strauss, S. Berger S. S., Volk, B. and Bauer J., Neurobiol. Aging, 17, 795-800 (1996).

13. Vane J. R. and Bottintg, R. M., Int. J. Tissue React., 20(1), 3-15 (1998)

14. SCRIP, No. 2527 p. 20 (2000).

15. McAdam, B. F., Catella-Lawson, F., Mardini, I. A., Kapoor, S. Lawson, J. A., and FitzGerald, G. A. Proc. Natl. Acad. Sci. USA, 96 272-277 (1999).

16. Hallenbeck, J. M., Acta Neurochir. Suppl. (Wien), 66, 27-31 (1996)

17. Beamer, N. B., Coull, B. M., Clark W. M., Hazel, J. S. and Silberger, J. R., Ann. Neurol., 37, 800-805 (1995).

18. Aktan, S. Aykut C., and Ercan, S., Prostagland. Leukot. Essent. Fatty Acids, 43, 247-249 (1991).

19. Tuor, U. I. and Del Bigio, M. R., Brain Res., 743, 258-262 (1996).

20. Tanaka et al. Neurochem. Res., 19, 117-122 (1994).

21. Tanaka et al. Neurochem. Res., 20, 663-667 (1995).

22. Bundgard, H. and Nielsen, N. M., Int. J. Pharmaceutics, 43, 101-110 (1988).

23. Liebeskind, L. S., Yu, M. S. and Fengl, R. W., J. Org. Chem., 58, 3543-3549 (1993)

24. Ellman, G C, Courtney, K D, Anders, V and Featherstone, R M, Biochem. Pharmacol., 7, 88-95 (1961).

25. Finney, D. J., in Statistical methods in biological assays, Charles Griffin & Co., London, 2nd edition, pp 524-530 (1964).

26. Burch, R. M., DeHaas, C., Arch. Pharmacol., 342, 189-193 (1990).

27. Gamache, D. A. and Ellis, E. F., J. Neurosurg, 65:686-692 (1986).

28. Chen, Y., Constantini, S., Trembovler, V., Weinstock, M., and Shohami, E., J. Neurotrauma, 13, 557-588 (1996).

29. Krieglstein, V. J. and Heuer, H., Arzneim-Forsch/Drug Res., 36 1568-1572 (1989).

30. Nakanishi, M., Yasuda, H. and Tsumagari, T., Life Sci., 13, 467-474 (1973).

31. Fitzgerald, B. B. and Costa, L. G., Toxicol. Appl. Pharmacol., 117, 122-125 (1992).

32. Pharmacological experiments on isolated preparations by the staff of the Department of Pharmacology University of Edinburgh, 2nd edition E. & S. Livingstone Edinburgh and London pp 64-66 (1970).

33. Whitehouse, M. W> and Rainsford, K. D., J. Pharmacol, 32, 795 (1980)

34. King, G. A., Arch Int. Pharmacodyn. Ther. 286, 282-298 (1987).

35. Singer, F., Mayrhofer, F., Klein, G., Hawel, R. and Kollenz, C. J., Int J. Clin. Pharmacol. Ther., 38, 15-24 (2000).

36. Geisslinger, G., Schuster, O., Stock, K. P., Loew, D., Bach, G. L. and Brune, K., Eur. J. Clin. Pharmacol., 38, 493-7 (1990).

37. Menzel-Soglowek, S., Geisslinger, G. and Brune, K., Agents Action Suppl., 44, 23-9 (1993).

What is claimed is:

1. A chimeric compound comprising a cholinergic up-regulator moiety and a non-steroidal anti-inflammatory moiety being covalently linked thereto.

2. The chimeric compound of claim 1, wherein said cholinergic up-regulator moiety and said non-steroidal anti-inflammatory moiety are covalently linked via a hydrocarbon spacer.

3. The chimeric compound of claim 2, wherein said non-steroidal anti-inflammatory moiety is covalently attached to said spacer via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to said C via a single covalent bond.

4. The chimeric compound of claim 3, wherein said bond is selected from the group consisting of an ester bond and an amide bond.

5. The chimeric compound of claim 4, wherein said ester bond is selected from the group consisting of a carboxylic ester bond and a glycol amide ester bond.

6. The chimeric compound of claim 3, wherein said bond is hydrolizable by a brain derived esterase.

7. The chimeric compound of claim 3, wherein said bond is hydrolizable by a brain derived amidase.

8. The chimeric compound of claim 2, wherein said hydrocarbon spacer comprises at least one hydrocarbon selected from the group consisting of an alkyl having 2-20 carbon atoms, a cycloalkyl having 3-20 carbon atoms and an aryl having 6-20 carbon atoms.

9. The chimeric compound of claim 1, wherein said cholinergic up-regulator moiety is selected from the group consisting of a cholinesterase inhibitor residue, a nicotinic receptor agonist residue and a muscarinic receptor agonist residue.

10. The chimeric compound of claim 9, wherein said cholinesterase inhibitor residue is a pyridostigmine residue.

11. The chimeric compound of claim 10, wherein said pyridostigmine residue is a 3-N,N-dimethylcarbamoyl pyridinium bromide residue.

12. The chimeric compound of claim 9, wherein said nicotinic agonist residue is selected from the group consisting of a nicotine residue and a cytisine residue.

13. The chimeric compound of claim 9, wherein said muscarinic receptor agonist residue is selected from the group consisting of an arecoline residue and a pilocarpine residue.

14. The chimeric compound of claim 1, wherein said non-steroidal anti-inflammatory moiety comprises a residue of a non-steroidal anti-inflammatory drug characterized by a functional group selected from the group consisting of a free carboxylic acid group and a free amine group.

15. The chimeric compound of claim 14, wherein said non-steroidal anti-inflammatory moiety is selected from the group consisting of an ibuprofen residue, an indomethacin residue, a naproxen residue, a diclofenac residue and an aspirin residue.

16. The chimeric compound of claim 15, wherein said ibuprofen residue is selected from the group consisting of an (±)-ibuprofen residue, S-(+)-ibuprofen residue and R-(−)-ibuprofen residue.

17. The chimeric compound of claim 1, characterized by lipophilicity sufficient for permitting the compound to cross a blood brain barrier of an organism.

18. A chimeric compound of a general formula:
A-S-B
wherein:
A is a cholinergic up-regulator moiety selected from the group consisting of a cholinesterase inhibitor residue, a nicotinic receptor agonist residue and a muscarinic receptor agonist residue;
B is a non-steroidal anti-inflammatory moiety characterized by a functional group selected from the group consisting of a free carboxylic acid group and a free amine group; and
S is a hydrocarbon spacer being covalently linked to B via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to said C via a single covalent bond.

19. The chimeric compound of claim 18, wherein said cholinesterase inhibitor residue is a pyridostigmine residue.

20. The chimeric compound of claim 19, wherein said pyridostigmine residue is a 3-N,N-dimethylcarbamoyl pyridinium bromide residue.

21. The chimeric compound of claim 18, wherein said nicotinic agonist residue is selected from the group consisting of a nicotine residue and a cytisine residue.

22. The chimeric compound of claim 18, wherein said muscarinic receptor agonist residue is selected from the group consisting of an arecoline residue and a pilocarpine residue.

23. The chimeric compound of claim 18, wherein said non-steroidal anti-inflammatory moiety is selected from the group consisting of an ibuprofen residue, an indomethacin residue, a naproxen residue, a diclofenac residue and an aspirin residue.

24. The chimeric compound of claim 23, wherein said ibuprofen residue is selected from the group consisting of an (±)-ibuprofen residue, a S-(+)-ibuprofen residue and a R-(−)-ibuprofen.

25. The chimeric compound of claim 18, wherein said bond is selected from the group consisting of an ester bond and an amide bond.

26. The chimeric compound of claim 25, wherein said ester bond is selected from the group consisting of a carboxylic ester bond and a glycol amide ester bond.

27. The chimeric compound of claim 18, wherein said hydrocarbon spacer comprises at least one hydrocarbon selected from the group consisting of an alkyl having 2-20 carbon atoms, a cycloalkyl having 3-20 carbon atoms and aryl having 6-20 carbon atoms.

28. A pharmaceutical composition comprising, as an active ingredient, the compound of claim 1, and a pharmaceutically acceptable carrier.

29. The pharmaceutical composition of claim 28, wherein said cholinergic up-regulator moiety and said non-steroidal anti-inflammatory moiety are covalently linked via a hydrocarbon spacer.

30. The pharmaceutical composition of claim 29, wherein said non-steroidal anti-inflammatory moiety is covalently attached to said spacer via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to said C via a single covalent bond.

31. The pharmaceutical composition of claim 30, wherein said bond is selected from the group consisting of an ester bond and an amide bond.

32. The pharmaceutical composition of claim 31, wherein said ester bond is selected from the group consisting of a carboxylic ester bond and a glycol amide ester bond.

33. The pharmaceutical composition of claim 30, wherein said bond is hydrolizable by a brain derived esterase.

34. The pharmaceutical composition of claim 30, wherein said bond is hydrolizable by a brain derived amidase.

35. The pharmaceutical composition of claim 29, wherein said hydrocarbon spacer comprises at least one hydrocarbon selected from the group consisting of an alkyl having 2-20 carbon atoms, a cycloalkyl having 3-20 carbon atoms and an aryl having 6-20 carbon atoms.

36. The pharmaceutical composition of claim 28, wherein said cholinergic up-regulator moiety is selected from the group consisting of a cholinesterase inhibitor residue, a nicotinic receptor agonist residue and a muscarinic receptor agonist residue.

37. The pharmaceutical composition of claim 36, wherein said cholinesterase inhibitor residue is a pyridostigmine residue.

38. The pharmaceutical composition of claim 37, wherein said pyridostigmine residue is a 3-N,N-dimethylcarbamoyl pyridinium bromide residue.

39. The pharmaceutical composition of claim 36, wherein said nicotinic agonist residue is selected from the group consisting of a nicotine residue and a cytisine residue.

40. The pharmaceutical composition of claim 36, wherein said muscarinic receptor agonist residue is selected from the group consisting of an arecoline residue and a pilocarpine residue.

41. The pharmaceutical composition of claim 28, wherein said non-steroidal anti-inflammatory moiety comprises a residue of a non-steroidal anti-inflammatory drug characterized by a functional group selected from the group consisting of a free carboxylic acid group and a free amine group.

42. The pharmaceutical composition of claim 41, wherein said non-steroidal anti-inflammatory moiety is selected from the group consisting of an ibuprofen residue, an indomethacin residue, a naproxen residue, a diclofenac residue and an aspirin residue.

43. The pharmaceutical composition of claim 42, wherein said ibuprofen residue is selected from the group consisting of an (±)-ibuprofen residue, S-(+)-ibuprofen residue and R-(−)-ibuprofen residue.

44. The pharmaceutical composition of claim 28, characterized by lipophilicity sufficient for permitting the compound to cross a blood brain barrier of an organism.

45. The pharmaceutical composition of claim 28, formulated for transdermal delivery.

46. The pharmaceutical composition of claim 28, formulated for nasal administration.

47. The pharmaceutical composition of claim 28, formulated for administration by inhalation.

48. The pharmaceutical composition of claim 28, formulated for administration by injection.

49. A pharmaceutical composition comprising, as an active ingredient, the compound of claim 18, and a pharmaceutically acceptable carrier.

50. The pharmaceutical composition of claim 49, wherein said cholinergic up-regulator moiety and said non-steroidal anti-inflammatory moiety are covalently linked via a hydrocarbon spacer.

51. The pharmaceutical composition of claim 50, wherein said non-steroidal anti-inflammatory moiety is covalently attached to said spacer via a —C(═X)Y— bond, where X is a non-substituted or substituted oxygen, sulfur or nitrogen atom and Y is a substituted or non-substituted carbon, oxygen, nitrogen, sulfur, silicon or phosphor atom linked to said C via a single covalent bond.

52. The pharmaceutical composition of claim 51, wherein said bond is selected from the group consisting of an ester bond and an amide bond.

53. The pharmaceutical composition of claim 52, wherein said ester bond is selected from the group consisting of a carboxylic ester bond and a glycol amide ester bond.

54. The pharmaceutical composition of claim 51, wherein said bond is hydrolizable by a brain derived esterase.

55. The pharmaceutical composition of claim 51, wherein said bond is hydrolizable by a brain derived amidase.

56. The pharmaceutical composition of claim 50, wherein said hydrocarbon spacer comprises at least one hydrocarbon selected from the group consisting of an alkyl having 2-20 carbon atoms, a cycloalkyl having 3-20 carbon atoms and an aryl having 6-20 carbon atoms.

57. The pharmaceutical composition of claim 49, wherein said cholinergic up-regulator moiety is selected from the group consisting of a cholinesterase inhibitor residue, a nicotinic receptor agonist residue and a muscarinic receptor agonist residue.

58. The pharmaceutical composition of claim 57, wherein said cholinesterase inhibitor residue is a pyridostigmine residue.

59. The pharmaceutical composition of claim 58, wherein said pyridostigmine residue is a 3-N,N-dimethylcarbamoyl pyridinium bromide residue.

60. The pharmaceutical composition of claim 57, wherein said nicotinic agonist residue is selected from the group consisting of a nicotine residue and a cytisine residue.

61. The pharmaceutical composition of claim 57, wherein said muscarinic receptor agonist residue is selected from the group consisting of an arecoline residue and a pilocarpine residue.

62. The pharmaceutical composition of claim 49, wherein said non-steroidal anti-inflammatory moiety comprises a residue of a non-steroidal anti-inflammatory drug characterized by a functional group selected from the group consisting of a free carboxylic acid group and a free amine group.

63. The pharmaceutical composition of claim 62, wherein said non-steroidal anti-inflammatory moiety is selected from the group consisting of an ibuprofen residue, an indomethacin residue, a naproxen residue, a diclofenac residue and an aspirin residue.

64. The pharmaceutical composition of claim 63, wherein said ibuprofen residue is selected from the group consisting of an (±)-ibuprofen residue, S-(+)-ibuprofen residue and R-(−)-ibuprofen residue.

65. The pharmaceutical composition of claim 49, characterized by lipophilicity sufficient for permitting the compound to cross a blood brain barrier of an organism.

66. The pharmaceutical composition of claim 49, formulated for transdermal delivery.

67. The pharmaceutical composition of claim 49, formulated for nasal administration.

68. The pharmaceutical composition of claim 49, formulated for administration by inhalation.

69. The pharmaceutical composition of claim 49, formulated for administration by injection.

70. A method of synthesizing the chimeric compound of claim 1, the method comprising the steps of:
(a) converting a non-steroidal anti-inflammatory drug into a non-steroidal anti-inflammatory-ester, including a hydrocarbon chain terminating with a reactive halide group; and
(b) reacting said non-steroidal anti-inflammatory-ester including said hydrocarbon chain terminating with said reactive halide group with a cholinergic up-regulator, so as to obtain the chimeric compound having said cholinergic up-regulator moiety covalently linked to said non-steroidal anti-inflammatory moiety via said hydrocarbon spacer.

71. The method of claim 70, wherein said hydrocarbon chain has at least one hydrocarbon selected from the group consisting of an alkyl having 2-20 carbon atoms, a cycloalkyl having 3-20 carbon atoms and an aryl having 6-20 carbon atoms.

72. The method of claim 70, wherein said non-steroidal anti-inflammatory drug is characterized by a functional group selected from the group consisting of a free carboxylic acid group and free amine group.

73. The method of claim 72, wherein said non-steroidal anti-inflammatory drug is selected from the group consisting of ibuprofen, indomethacin, naproxen, diclofenac and aspirin.

74. The method of claim 73, wherein said ibuprofen is selected from the group consisting of (±)-ibuprofen, S-(+)-ibuprofen and R-(−)-ibuprofen.

75. The method of claim 70, wherein said cholinergic up-regulator is selected from the group consisting of a cholinesterase inhibitor, a nicotinic agonist and a muscarinic agonist.

76. The method of claim 75, wherein said cholinesterase inhibitor is a pyridostigmine.

77. The method of claim 76, wherein said pyridostigmine is 3-N,N-dimethylcarbamoyl pyridinium bromide.

78. The method of claim 75, wherein said nicotinic agonist is selected from the group consisting of nicotine and cytisine.

79. The method of claim 75, wherein said muscarinic agonist is selected from the group consisting of arecoline and pilocarpine.

80. A method of synthesizing the chimeric compound of claim 1, the method comprising the steps of.
(a) converting a non-steroidal anti-inflammatory drug into a non-steroidal anti-inflammatory-amide, said amide including a hydrocarbon chain terminating with a reactive halide group; and
(b) reacting said non-steroidal anti-inflammatory-amide including said hydrocarbon chain terminating with said reactive halide group with a cholinergic up-regulator, so as to obtain the chimeric compound having said cholinergic up-regulator moiety covalently linked to said non-steroidal anti-inflammatory moiety via said hydrocarbon spacer.

81. The method of claim 80, wherein said hydrocarbon chain has at least one hydrocarbon selected from the group consisting of an alkyl having 2-20 carbon atoms, a cycloalkyl having 3-20 carbon atoms and an aryl having 6-20 carbon atoms.

82. The method of claim 80, wherein said non-steroidal anti-inflammatory drug is characterized by a functional group selected from the group consisting of a free carboxylic acid group and free amine group.

83. The method of claim 82, wherein said non-steroidal anti-inflammatory drug is selected from the group consisting of ibuprofen, indomethacin, naproxen, diclofenac and aspirin.

84. The method of claim 83, wherein said ibuprofen is selected from the group consisting of (±)-ibuprofen, S-(+)-ibuprofen and R-(−)-ibuprofen.

85. The method of claim 80, wherein said cholinergic up-regulator is selected from the group consisting of a cholinesterase inhibitor, a nicotinic agonist and a muscarinic agonist.

86. The method of claim 85, wherein said cholinesterase inhibitor is a pyridostigmine.

87. The method of claim 86, wherein said pyridostigmine is 3-N,N-dimethylcarbamoyl pyridinium bromide.

88. The method of claim 85, wherein said nicotinic agonist is selected from the group consisting of nicotine and cytisine.

89. The method of claim 85, wherein said muscarinic agonist is selected from the group consisting of arecoline and pilocarpine.

90. A method of synthesizing the chimeric compound of claim 1, the method comprising the steps of:
(a) converting a cholinergic up-regulator into its N(ring)-substituted derivative, said derivative including a hydrocarbon chain terminating with a reactive hydroxyl group; and
(b) reacting said N(ring)-substituted derivative including said hydrocarbon chain terminating with said reactive hydroxyl group with a reactive derivative of a non-steroidal anti-inflammatory drug, so as to obtain the chimeric compound having said cholinergic up-regulator moiety covalently linked to said non-steroidal anti-inflammatory moiety.

91. The method of claim 90, wherein said hydrocarbon chain has at least one hydrocarbon selected from the group consisting of an alkyl having 2-20 carbon atoms, a cycloalkyl having 3-20 carbon atoms and an aryl having 6-20 carbon atoms.

92. The method of claim 90, wherein said non-steroidal anti-inflammatory drug is characterized by a functional group selected from the group consisting of a free carboxylic acid group and a free amine group.

93. The method of claim 92, wherein said non-steroidal anti-inflammatory drug is selected from the group consisting of ibuprofen, indomethacin, naproxen, diclofenac and aspirin.

94. The method of claim 93, wherein said ibuprofen is selected from the group consisting of (±)-ibuprofen, S-(+)-ibuprofen and R-(−)-ibuprofen.

95. The method of claim 90, wherein said cholinergic up-regulator is selected from the group consisting of a cholinesterase inhibitor, a nicotinic agonist and a muscarinic agonist.

96. The method of claim 95, wherein said cholinesterase inhibitor is a pyridostigmine.

97. The method of claim 96, wherein said pyridostigmine is 3-N,N-dimethylcarbamoyl pyridinium bromide.

98. The method of claim 95, wherein said nicotinic agonist is selected from the group consisting of nicotine and cytisine.

99. The method of claim 95, wherein said muscarinic antagonist is selected from the group consisting of arecoline and pilocarpine.

100. The method of claim 90, further comprising the step of converting said N(ring)-substituted derivative including said hydrocarbon chain terminating with said reactive hydroxyl group into a tertiary amine N(ring)-substituted derivative including said hydrocarbon chain terminating with said reactive hydroxyl group, prior to said step (b).

101. A method of treating, ameliorating or preventing a central nervous system disorder or disease in an organism, the method comprising the step of administering to said organism a therapeutically effective amount of the compound of claim 1.

102. The method of claim 101, wherein said central nervous system disorder or disease is selected from the group consisting of Alzheimer's disease, cerebrovascular dementia, Parkinson's disease, basal ganglia degenerative diseases, motoneuron diseases, Scrapie, spongyform encephalopathy and Creutzfeldt-Jacob's disease.

103. The method of claim 101, wherein said central nervous system disorder or disease is selected from the group consisting of cerebral ischemia, transient hypoxia and stroke.

104. The method of claim 101, wherein said central nervous system disorder or disease is a result of a head injury.

105. The method of claim 101, wherein said central nervous system disorder or disease is accompanied by an inflammatory process.

106. The method of claim 105, wherein said inflammatory process is selected from the group consisting of an inflammatory process induced by infection, an inflammatory process induced by a tumor and an inflammatory process induced by post-operative brain edema.

107. The method of claim 106, wherein said infection is selected from the group consisting of viral infection and bacterial infection.

108. The method of claim 101, wherein said organism is a mammal.

109. The method of claim 101, wherein said mammal is a human being.

110. The chimeric compound of claim 1, wherein said cholinergic up-regulator moiety is selected from the group consisting of a reversible cholinesterase inhibitor residue and an irreversible cholinesterase inhibitor residue.

111. The chimeric compound of claim 18, wherein said cholinergic up-regulator moiety is selected from the group consisting of a reversible cholinesterase inhibitor residue and an irreversible cholinesterase inhibitor residue.

112. The pharmaceutical composition of claim 28, wherein said cholinergic up-regulator moiety is selected from the group consisting of a reversible cholinesterase inhibitor residue and an irreversible cholinesterase inhibitor residue.

113. The pharmaceutical composition of claim 49, wherein said cholinergic up-regulator moiety is selected from the group consisting of a reversible cholinesterase inhibitor residue and an irreversible cholinesterase inhibitor residue.

114. The method of claim 70, wherein said cholinergic up-regulator moiety is selected from the group consisting of a reversible cholinesterase inhibitor residue and an irreversible cholinesterase inhibitor residue.

115. The method of claim 80, wherein said cholinergic up-regulator moiety is selected from the group consisting of a reversible cholinesterase inhibitor residue and an irreversible cholinesterase inhibitor residue.

116. The method of claim 90, wherein said cholinergic up-regulator moiety is selected from the group consisting of a reversible cholinesterase inhibitor residue and an irreversible cholinesterase inhibitor residue.

117. A reversible cholinesterase inhibitor having a general formula A: 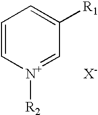
wherein,
R₁ is C(═Q)Z-R₃;
R₂ is selected from the group consisting of hydrogen, an alkyl, a hydroxyalkyl, a haloalkyl, an alkylamine, a cycloalkyl and an aryl;
X is a halide.
Q and Z are each independently selected from the group consisting of oxygen and sulfur; and
R₃ is selected from the group consisting of an alkyl, a cycloalkyl and an aryl.

118. The reversible cholinesterase inhibitor of claim 117, wherein Q and Z are each oxygen, R₃ is methyl, R₂ is alkyl and X is selected from the group consisting of bromide and iodide.

119. A method of synthesizing the reversible cholinesterase inhibitor of claim 117, comprising reacting a pyridine ring substituted at position 3 by said R₁ with a R₂ residue terminating with said X, so as to produce a quaternary pyridinium halide substituted at the N(ring) position by said R₂ and at position 3 by said R₁.

120. A method of treating, ameliorating or preventing a central nervous system disorder or disease in an organism, the method comprising the step of administering to said organism a therapeutically effective amount of the reversible cholinesterase inhibitor of claim 117.

121. The method of claim 120, wherein said central nervous system disorder or disease is selected from the group consisting of Alzheimer's disease, cerebrovascular dementia, Parkinson's disease, basal ganglia degenerative diseases, motoneuron diseases, Scrapie, spongyform encephalopathy and Creutzfeldt-Jacob's5 disease.

122. The method of claim 120, wherein said central nervous system disorder or disease is selected from the group consisting of cerebral ischemia, transient hypoxia and stroke.

123. The method of claim 120, wherein said central nervous system disorder or disease is a result of a head injury.

124. A reversible cholinesterase inhibitor having a general formula B: 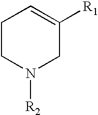
wherein,
R₁ is C(═Q)Z-R₃;
R₂ is selected from the group consisting of hydrogen, an alkyl, a hydroxyalkyl, a haloalkyl, an alkylamine, a cycloalkyl and an aryl;
Q and Z are each independently selected from the group consisting of oxygen or sulfur; and
R₃ is selected from the group consisting of an alkyl, a cycloalkyl and an aryl.

125. The reversible cholinesterase inhibitor of claim 124, wherein Q and Z are each oxygen, R₃ is methyl and R₂ is alkyl.

126. A method of synthesizing the reversible cholinesterase inhibitor of claim 124, comprising:
(a) reacting a pyridine ring substituted at position 3 by said R₁ with an organic halide and/or a reactive inorganic halide, so as to produce a quaternary pyridinium halide substituted by said R₁ at position 3; and
(b) reducing said quaternary pyridinium halide, so as to produce a tertiary tetrahydropyridine ring, substituted by said R₁ group at position 3.

127. The method of claim 126, wherein said reactive inorganic halide is potassium iodide.

128. The method of claim 126, wherein said organic halide is a R₂ residue terminating with a halide group and said quaternary pyridinium halide is further substituted at the N(ring) position by said R₂.

129. A method of treating, ameliorating or preventing a central nervous system disorder or disease in an organism, the method comprising the step of administering to said organism a therapeutically effective amount of the reversible cholinesterase inhibitor of claim 124.

130. The method of claim 129, wherein said central nervous system disorder or disease is selected from the group consisting of Alzheimer's disease, cerebrovascular dementia, Parkinson's disease, basal ganglia degenerative diseases, motoneuron diseases, Scrapie, spongyform encephalopathy and Creutzfeldt-Jacob's disease.

131. The method of claim 129, wherein said central nervous system disorder or disease is selected from the group consisting of cerebral ischemia, transient hypoxia and stroke.

132. The method of claim 129, wherein said central nervous system disorder or disease is a result of a head injury.

133. The chimeric compound of claim 1, characterized by cholinergic up-regulation activity and inflammation down-regulation activity exerted by said chimeric compound and by hydrolytic derivatives thereof.
